(19) United States
(12) Patent Application Publication
(10) Pub. No.: US 2025/0025465 A1
(43) Pub. Date: Jan. 23, 2025

(54) WT-IDH1 INHIBITION USING A COVALENT AND BRAIN-PENETRANT SMALL MOLECULAR INHIBITOR FOR FERROPTOSIS INDUCTION IN HIGH-GRADE GLIOMA

(71) Applicant: Northwestern University, Evanston, IL (US)

(72) Inventors: Kevin Murnan, Evanston, IL (US); Serena Tommasini Ghelfi, Evanston, IL (US); Lisa Agnes Hurley, Evanston, IL (US); Alexander H. Stegh, Evanston, IL (US)

(21) Appl. No.: 18/711,276

(22) PCT Filed: Nov. 17, 2022
(86) PCT No.: PCT/US2022/080079

§ 371 Date: May 17, 2024

Related U.S. Application Data
(60) Provisional application No. 63/264,239, filed on Nov. 17, 2021.

(51) Int. Cl. A61K 31/517; A61K 45/06; A61P 35/00

(52) U.S. Cl. CPC A61K 31/517; A61K 45/06; A61P 35/00

(57) ABSTRACT

Disclosed are methods of treating diseases or disorders associated with the expression of wild type isocitrate dehydrogenase 1 (IDH1). The disclosed methods may be utilized to treat diseases or disorders associated with cell proliferation, including cancer.

### CROSS-REFERENCE TO RELATED APPLICATIONS

This application claims benefit of priority to U.S. Patent Application Ser. No. 63/264,239, filed Nov. 17, 2021, the contents of which are incorporated by reference in its entirety.

### STATEMENT REGARDING FEDERALLY SPONSORED RESEARCH OR DEVELOPMENT

This invention was made with government support under P50CA221747 awarded by the National Institutes of Health. The government has certain rights in the invention.

### SEQUENCE LISTING

A Sequence Listing accompanies this application and is submitted as an xml file of the sequence listing named “702581_02234.xml” which is 4,394 bytes in size and was created on Nov. 16, 2022. The sequence listing is electronically submitted via Patent Center and is incorporated herein by reference in its entirety.

### BACKGROUND

Progress in the biochemical characterization of high-grade glioma (HGG) identified profound shifts in how HGG tumor cells utilize nutrients, including and especially fatty acids. Increased de novo synthesis, mobilization and uptake of fatty acids are required to provide sufficient lipids for membrane biogenesis in support of rapid tumor cell division and growth. In addition to their structural roles as components of the plasma membrane, fatty acid-derived lipids regulate ferroptotic cell death when oxidized by iron-dependent lipoxygenase enzymes. Ferroptosis is a type of programmed cell death controlled by environmental, signaling and genetic inputs, including radiation, redox homeostasis and metabolism. While the induction of ferroptosis has emerged as a potent tumor suppressive mechanism, challenges in the field relate to the identification of molecular events that link specific genetic aberration to the regulation of ferroptosis, and the development of ferroptosis-based therapies for clinical use.

### SUMMARY

Disclosed herein are compounds and methods of treating cell proliferative diseases. The method may comprise administering to the subject a compound with the formula:

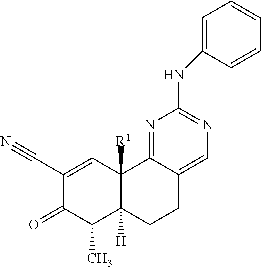

wherein R¹ is selected from methyl or phenyl. The cell proliferative disease may be a cancer, such as a brain cancer. Exemplary cell proliferative include astrocytoma, oligodendroglioma, mixed glioma, and ependymoma. In some embodiments, the subject is in need for a treatment for high grade glioma (HGG).

Another aspect of the technology provides for a method for treating a subject in need of treatment for a disease or disorder associated with expression of wild type isocitrate dehydrogenase 1 (IDH1). The method may comprise administering to the subject an effective amount of a therapeutic agent that inhibits the biological activity of IDH1. The subject may be administered any of the compounds disclosed herein. The disease or disorder associated with expression of wild type isocitrate dehydrogenase 1 (IDH1) may be a cell proliferative disease, such as a cancer. Exemplary cell proliferative include astrocytoma, oligodendroglioma, mixed glioma, and ependymoma. In some embodiments, the subject is in need for a treatment for high grade glioma (HGG).

### BRIEF DESCRIPTION OF THE FIGURES

FIG. 1. wt-IDH1 is over-expressed in HGG. (A). IDH1 mRNA expression in TCGA HGG tumors. (B). TCGA patient survival in wt-IDH1low low vs. wt-IDH1high groups. (C). IDH1 mRNA expression in grade II, III and IV glioma. (D) Representative wt-IDH1 IHC and H&E staining of specimens from a HGG TMA. (E). Quantification of wt-IDH1 staining intensities by IHC score. Bar, 50 μm. *p<0.05; **p<0.0001; ***p<0.00005.

FIG. 2. wt-IDH1 promotes HGG progression in vivo. Quantification of bioluminescence and survival of HGG PDX-bearing mice, expressing Co and shIDH1 (A1, A2), and p53 and PTEN co-deleted neural stem cells expressing CSII and CSII-wt-IDH1 (B1, B2). Histograms in panels A and B show mean±SEM. (C-D) Kaplan-Meyer tumor-free survival analysis of loss- and gain-of-function GEMM cohorts on tamoxifen. *p<0.05

FIG. 3. Knockdown of wt-IDH1 in decreases alpha-ketoglutarate, NADPH, and fatty acid levels. (A) Levels of alpha-ketoglutarate. (B) NADPH/NADP+ ratio. (C) Levels of SFA and MUFAs determined by LC-MS. (D) Levels of total and 13C-labeled SFAs and MUFAs in GICs labeled with 13C-glucose or acetate tracers. (E) Levels of acetyl-CoA labeled with 13C-acetate. Shown is the mean+/−standard deviation. *p<0.05.

FIG. 4. Increased lipid droplet formation in wt-IDH1high HGG, as determined by Oil Red O staining on PDX tumor sections.

FIG. 5. wt-IDH1 inactivation enhances RT anti-tumor effect (A). RT enhancement ratio, as assessed via clonogenic growth assay in U87MG cells, upon genetic wt-IDH1inactivation, in the presence or absence of the ROS scavenger N-acetyl cytosine (NAC). (B). wt-IDH1 in doxy-treated xenogafts. (C). Tumor volume in mice subcutaneously implanted with U87MG cells carrying a doxy-inducible shIDH1 were treated with doxy and irradiated with a total of 14 Gy administered in 7 fractions of 2 Gy, and tumor volumes were measured. *p<0.05. (D.) Kaplan-Meyer estimates for tumor tripling, *p<0.005 vs. control; **p<0.001 vs. control, RT and doxy.

FIG. 6. wt-IDH1i-13 is a selective covalent wt-IDH1 inhibitor. NADPH production by recombinant wt-IDH2 as a function of compound concentrations using indicated active or inactive wt-IDH1 inhibitors.

FIG. 7. X-ray crystal structures of wt-IDH1i-13 with wt-IDH1. The binding site of one monomer of the wt-IDH1 complex is shown with key protein residues shown in sticks. H-bonds are illustrated by dashed red lines (PDB code: 6BL1) (See Jakob et al., “Novel Modes of Inhibitor of Wild-Type Isocitrate Dehydrogenase 1 (IDH1): Direct Covalent Modification of His315,” J. Med. Chem. 2018, 61, 15, 6647-6657, the content of which is incorporated herein by reference in its entirety).

FIG. 8. Model: wt-IDH1 inhibits ferroptosis through increased MUFA production and increased NAPDH-driven GPX4 lipid repair.

FIG. 9. wt-IDH1 controls lipid repair. (A-C). Knockdown wt-IDH1 increases ROS, decreases GSH and enhances MDA (PUFA peroxide) levels. (D). Knockdown of wt-DH1 reduces GSR activity. (E-F). Knockdown of wt-IDH1 reduces while overexpression of wt-IDH1 increases GPX4 activity. (G-I) GPX4 mRNA and protein levels are not regulated by wt-IDH1, as determined by RT-qPCR, western blotting, and analysis of the TCGA GBM dataset. *p<0.05.

FIG. 10. wt-IDH1 controls cell death induced by GPX4i RSL3 and RT. (A). wt-IDH1 knockdown cooperates with GPX4 inhibition. (B). wt-IDH1 overexpression blocks RT+GPX4i-induced cell death. *p<0.05.

FIG. 11. wt-DIH1i-13 promotes ferroptosis. SYTOX green staining of glioma cells co-treated with wt-IDH1i-13 plus ferrostatin (A), NAC (B) or alpha-ketoglutarate (α-KG) (C). *p<0.05.

FIG. 12. wt-DIH1i-13 enhances RT anti-tumor effect in vivo. (A). Treatment schedule. (B). Bioluminescence of an adult HGG PDX model treated with vehicle, RT (2 Gy x 5,M-F), 13i, or RT+13i (i.p. 10 mg/kg, M-F). (C). Kaplan-Meyer survival benefit analysis. (D) H&E stainings of normal brain from PDX mice treated with RT+13i. *p<0.05.

FIG. 13. Wt-IDH1 is overexpressed in and promotes the progression of other cancers. (A) TCGA dataset analysis of wt-IDH1 mRNA in lung adenocarcinoma (n=488) and lung squamous cell carcinoma (n=489) compared to normal lung tissue (n=50). (B) wt-IDH1 mRNA expression in AILD (n=6), ALCL (n=6), and PTCL (n=28) in comparison to CD4 (n=5), CD8 (n=5) or HLA-DRpositive T cells (n=10), and in DLBCL (n=73) in comparison to Foll (n=38), Germ (n=10), Mem (n=5) or naive B cells (n=5). (C) The diffuse large B cell lymphoma cell line SUDHL4 was lentivirally transduced with pLKO or shRNAs targeted to wt-IDH1, and wt-IDH1protein levels were assessed by western blotting. (D) Annexin V positivity (3 cultures per group; Mean±SD), (E) α-KG (1 culture per group; Mean±SD), (F) GSH (1 culture per group; Mean±SD), and (G) ROS levels (3 cultures per group; Mean±SD) were quantified in vehicle (Veh)- or ibrutinib-treated pLKO and shIDH1 cultures. (H, I) Bioluminescence of SUDHL4 pLKO and shIDH1 cells 14 days after flank implantation in SCID mice (n=5 animals per group; Mean±SEM). *p<5×10−10, **p<1×10−5, ***p<0.005, ****p<0.05, *****p<0.001, #p<0.01, ##p<0.0005. AILD, angioimmunoblastic lymphadenopathy; ALCL, anaplastic large cell lymphoma; PTCL, peripheral T-cell lymphoma; DLBCL, diffuse large B cell lymphoma; Foll, follicular; Germ, germinal; Mem, memory.

FIG. 14. Wt-IDH1 through enhanced α-KG production increases the activity of α-KG-dependent demethylases and in so doing, elevates IGFBP2 expression. IGFBP2 in turn recruits monocyte-derived macrophages and promotes their M2 alternative activation in support of tumor growth.

FIG. 15. GAGE analysis for various types of immune cells as a function of IDH1-wt expression in TCGA GBM.

FIG. 16. TAM content in murine tumors with IDH1-wt increase and loss-of-function. (A) Innate immune cells in IDH1-wt vs. vector engraftment tumors. (B-D) TAM, CD8+ T cell content and CD8⁺/TAM ratio in CT2A.IDH1-wt^{high} versus vector controls. Mean+/−SD; n=3. (E) TAM content in our GEM loss-of-function model. Mean+/−SD; n=3. *p<0.05.

FIG. 17. IDH1-wt induces IGFBP2 mRNA and protein. (A) Cytokine profiling of GICs in vitro and serum from PDX-bearing mice in vivo. (B) Densitometric quantification of cytokine abundance in serum [fold change (FC): IDH1-wt vs. CSII-expressing PDX]. (C) IGFBP2 mRNA level in GICs with IDH1-wt over-and underexpression. Mean+/−SEM; n=3. (D-E) IGFBP2 mRNA expression in normal brain tissue, and in IDH1-wt versus IDH1-mt TCGA GBM tumors. (F) Correlation between IDH1-wt and IGFBP2 transcript level in TCGA GBM. (G) TCGA patient survival in IGFBP2-wt^{low} versus IGFBP2^{high} groups.

FIG. 18. IDH1-wt controls IGFBP2 transcript through αKG-dependent demethylation. (A) α-KG level in control and IDH1-wt KD cells. Mean+/−SD; n=3. (B-C) RT-qPCR to assess IGFBP2 mRNA in shCo and IDH1-wt KD GICs+/−decitabine (DEA) or α-KG. Mean+/−SD; n=3. (D-E) Correlation analysis of methylation β value at 2 CpG sites within the IGFBP2 gene as a function of IGFBP2 and IDH1-wt mRNA level. *p<0.05.

### DETAILED DESCRIPTION

The present invention is described herein using several definitions, as set forth below and throughout the application.

Progress in the biochemical characterization of high-grade glioma (HGG) identified profound shifts in how HGG tumor cells utilize nutrients, including, and especially, fatty acids. Increased de novo synthesis, mobilization and uptake of fatty acids are required to provide sufficient lipids for membrane biogenesis in support of rapid tumor cell division and growth. In addition to their structural roles as components of the plasma membrane, fatty acid-derived lipids regulate ferroptotic cell death when oxidized by iron-dependent lipoxygenase enzymes. Ferroptosis is a type of programmed cell death controlled by environmental, signaling and genetic inputs, including radiation, redox homeostasis and metabolism. While the induction of ferroptosis has emerged as a potent tumor suppressive mechanism, challenges in the field relate to the identification of molecular events that link specific genetic aberration to the regulation of ferroptosis, and the development of ferroptosis-based therapies for clinical use.

De novo lipogenesis and the defense against oxidative lipid damage require large amounts of cytosolic NADPH. HGG up-regulate wild-type isocitrate dehydrogenase 1 (IDH1), referred to hereafter as ‘wt-IDH1high HGG’, to generate large quantities of cytosolic NADPH (Calvert et al., Cell reports, 2017). wt-IDH1 is a cytosolic and peroxisomal enzyme that catalyzes the oxidative decarboxylation of isocitrate to alpha-ketoglutarate, converting NADP+ to NADPH. RNAi-mediated knockdown of wt-IDH1, alone and in combination with radiation therapy (RT), slows the growth of patient-derived HGG xenografts, while overexpression of wt-IDH1 promotes intracranial HGG growth. Molecularly, wt-IDH1high HGG produce excess NADPH, which serves as a rate-limiting reductant that drives de novo fatty acid biosynthesis. Isotope tracer and liquid chromatography-based lipidomic studies indicated that wt-IDH1 supports the de novo biosynthesis of mono-unsaturated fatty acids (MUFAs) and promotes the incorporation of monounsaturated phospholipids into cell membrane. In addition, enhanced NADPH production in wt-IDH1high HGG increases glutathione (GSH) level, reduces reactive oxygen species (ROS), activates the phospholipid peroxidase glutathione peroxidase 4 (GPX4)-driven lipid repair pathway, and dampens the accumulation of polyunsaturated fatty acid (PUFA)-containing lipid peroxides, known executioners of ferroptosis. These findings support elevated wt-IDH1 expression, the ensuing increase in de novo fatty acid biosynthesis and protection against lipid peroxidation as metabolic contributors to HGG tumor growth and therapy resistance.

A therapeutic strategy for the pharmacological inhibition of wt-IDH1 has been devised. The Examples demonstrate the use and characterization of 13i, a first-in-class competitive alpha/beta-unsaturated enone (see Jakob et al., “Novel Modes of Inhibition of Wild-Type Isocitrate Dehydrogenase 1 (IDH1): Direct Covalent Modification of His315”, J Med Chem 61, 6647-6657). 13i potently inhibits wt-IDH1 enzymatic activity, by covalently binding to the NADP+ binding pocket, thereby preventing substrate access. The data indicates that 13i blocks wt-IDH1 with an EC50 of 14 nM and a cellular IC50 of 0.8-1.7 uM. On a molecular level, 13i promotes ferroptosis, which can be rescued by pre-treatment of cells with the peroxyl scavenger and ferroptosis inhibitor ferrostatin. 13i is brain-penetrant (see Chung et al., Cancer 2020), and like genetic ablation of IDH1, reduces the progression and extends the survival of wt-IDH1high GBM in combination with radiation therapy (RT).

### Definitions

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

A “subject in need thereof” as utilized herein may refer to a subject in need of treatment for a disease or disorder associated with isocitrate dehydrogenase activity and/or expression. A subject in need thereof may include a subject having a cell proliferative disease or disorder that is characterized by the activity and/or expression of IDH1. A subject in need thereof may include a subject having a cancer that is treated by administering a therapeutic agent that inhibits the biological activity of IDH1.

The term “subject” may be used interchangeably with the terms “individual” and “patient” and includes human and non-human mammalian subjects.

The disclosed methods may be utilized to treat diseases and disorders associated with IDH1 activity and/or expression which may include, but are not limited to cell proliferative diseases and diseases and disorders such as cancers. Suitable cancers for treatment by the disclosed methods may include, but are not limited to brain cancer, for example, astrocytoma, oligodendroglioma, mixed glioma, and ependymoma.

As used herein, “brain cancer” refers to an abnormal growth of cells, or tumors, in the brain. In some embodiments, brain cancer comprises a variety of cell proliferative diseases defined by their presence inside the skull. For example, brain cancer may refer to tumors that originated from cells of the brain, e.g., astrocytes, neurons, glial cells, oligodendrocytes, etc., or brain cancer may refer to cells with origins outside of the brain that have metastasized to the brain. In some embodiments, brain cancer comprises astrocytoma, oligodendroglioma, mixed glioma, and ependymoma. As used herein, “astrocytoma” is a cancer of astrocytes. The most aggressive astrocytoma is a glioblastoma, which is also called a glioblastoma multiforme. As used herein, “oligodendroglioma” refers to cancer of oligodendrocytes. As used herein, “mixed glioma” refers to cancer of both astrocytes and oligodendrocytes. As used herein, “ependymoma” refers to cancer of the cells lining the ventricles of the brain and the canal of the spinal cord. As used herein, “high grade glioma” refers to grade III or grade IV tumors. Grade III gliomas include anaplastic astrocytomas and anaplastic oligodendrogliomas. Grade IV gliomas are called glioblastomas. High-grade gliomas grow rapidly and can easily spread throughout the brain. These are the most aggressive types of glioma and are life-threatening.

As used herein, “ferroptosis” refers to a nonapoptotic, iron-dependent form of cell death that can be activated in cancer cells by natural stimuli and synthetic agents. Three essential hallmarks define ferroptosis, namely: the loss of lipid peroxide repair capacity by the phospholipid hydroperoxidase GPX4, the availability of redox-active iron, and oxidation of polyunsaturated fatty acid (PUFA)-containing phospholipids. Several processes including RAS/MAPK signaling, amino acid and iron metabolism, ferritinophagy, epithelial-to-mesenchymal transition, cell adhesion, and mevalonate and phospholipid biosynthesis can modulate susceptibility to ferroptosis. Ferroptosis sensitivity is also governed by p53 and KEAP1/NRF2 activity, linking ferroptosis to the function of key tumor suppressor pathways.

As used herein, “radiation therapy” or “radiotherapy” refers to directing a beam of high energy particles such as electrons, protons, or heavy ions into a target volume (e.g., a tumor or lesion) in a patient. Before the patient is treated with radiation, a treatment plan specific to that patient is developed. The plan defines various aspects of the radiotherapy using simulations and optimizations based on past experiences. For example, for intensity modulated particle therapy (IMPT), the plan can specify the appropriate beam type and the appropriate beam energy. Other parts of the plan can specify, for example, the angle of the beam relative to the patient/target volume, the beam shape, and the like. In general, the purpose of the treatment plan is to deliver sufficient radiation to the target volume while minimizing the exposure of surrounding healthy tissue to radiation.

The disclosed compounds may be utilized to modulate the biological activity of IDH1, including modulating the dehydrogenase activity of IDH1. The term “modulate” should be interpreted broadly to include “inhibiting” IDH1 biological activity including dehydrogenase activity.

IDH1 has been shown to have enzyme activities that include catalyzing the oxidative decarboxylation of isocitrate to form 2-oxoglutarate (a-ketoglutarate). IDH1 has ENZYME entry: EC 1.1.1.42. The compounds used in the methods disclosed herein may inhibit one or more of the activities of IDH1 accordingly.

Human isocitrate dehydrogenase 1 (IDH1) (SEQ ID NO: 1) has the amino acid sequence:

| | |
|---|---|
| MSKKISGGSV VEMQGDEMTR IIWELIKEKL IFPYVELDLH SYDLGIENRD ATNDQVTKDA | 60 |
| AEAIKKHNVG VKCATITPDE KRVEEFKLKQ MWKSPNGTIR NILGGTVFRE AIICKNIPRL | 120 |
| VSGWVKPIII GRHAYGDQYR ATDFVVPGPG KVEITYTPSD GTQKVTYLVH NFEEGGGVAM | 180 |
| GMYNQDKSIE DFAHSSFOMA LSKGWPLYLS TKNTILKKYD GRFKDIFQEI YDKQYKSQFE | 240 |
| AQKIWYEHRL IDDMVAQAMK SEGGFIWACK NYDGDVQSDS VAQGYGSLGM MTSVLVCPDG | 300 |
| KTVEAEAAHG TVTRHYRMYQ KGQETSTNPI ASIFAWTRGL AHRAKLDNNK ELAFFANALE | 360 |
| EVSIETIEAG FMTKDLAACI KGLPNVQRSD YLNTFEFMDK LGENLKIKLA QAKL | 414 |

Human IDH1 (SEQ ID NO: 2) has the nucleotide sequence:

| | |
|---|---|
| ctcatttttc cctacgtgga attggatcta catagctatg atttaggcat agagaatcgt | 60 |
| gatgccacca acgaccaagt caccaaggat gctgcagaag ctataaagaa gcataatgtt | 120 |
| ggcgtcaaat gtgccactat cactcctgat gagaagaggg ttgaggagtt caagttgaaa | 180 |
| caaatgtgga aatcaccaaa tggcaccata cgaaatattc tgggtggcac ggtcttcaga | 240 |
| gaagccatta tctgcaaaaa tatcccccgg cttgtgagtg gatgggtaaa acctatcatc | 300 |
| ataggtcgtc atgcttatgg ggatcaatac agagcaactg attttgttgt tcctgggcct | 360 |
| ggaaaagtag agataaccta cacaccaagt gacggaaccc aaaaggtgac atacctggta | 420 |
| cataactttg aagaaggtgg tggtgttgcc atggggatgt ataatcaaga taagtcaatt | 480 |
| gaagattttg cacacagttc cttccaaatg gctctgtcta agggttggcc tttgtatctg | 540 |
| agcaccaaaa acactattct gaagaaatat gatgggcgtt ttaaagacat ctttcaggag | 600 |
| atatatgaca agcagtacaa gtcccagttt gaagctcaaa agatctggta tgagcatagg | 660 |
| ctcatcgacg acatggtggc ccaagctatg aaatcagagg gaggcttcat ctgggcctgt | 720 |
| aaaaactatg atggtgacgt gcagtcggac tctgtggccc aagggtatgg ctctctcggc | 780 |
| atgatgacca gcgtgctggt ttgtccagat ggcaagacag tagaagcaga ggctgcccac | 840 |
| gggactgtaa cccgtcacta ccgcatgtac cagaaaggac aggagacgtc caccaatccc | 900 |
| attgcttcca tttttgcctg gaccagaggg ttagcccaca gagcaaagct tgataacaat | 960 |
| aaagagcttg ccttctttgc aaatgctttg gaagaagtct ctattgagac aattgaggct | 1020 |
| ggcttcatga ccaaggactt ggctgcttgc attaaaggtt tacccaatgt gcaacgttct | 1080 |
| gactacttga atacatttga gttcatggat aaa | 1113 |

### Pharmaceutical Compositions

The compounds employed in the compositions and methods disclosed herein may be administered as pharmaceutical compositions and, therefore, pharmaceutical compositions incorporating the compounds are considered to be embodiments of the compositions disclosed herein. Such compositions may take any physical form which is pharmaceutically acceptable; illustratively, they can be orally administered pharmaceutical compositions. Such pharmaceutical compositions contain an effective amount of a disclosed compound, which effective amount is related to the daily dose of the compound to be administered. Each dosage unit may contain the daily dose of a given compound or each dosage unit may contain a fraction of the daily dose, such as one-half or one-third of the dose. The amount of each compound to be contained in each dosage unit can depend, in part, on the identity of the particular compound chosen for the therapy and other factors, such as the indication for which it is given. The pharmaceutical compositions disclosed herein may be formulated so as to provide quick, sustained, or delayed release of the active ingredient after administration to the patient by employing well known procedures.

The compounds for use according to the methods of disclosed herein may be administered as a single compound or a combination of compounds. For example, a compound that inhibits the biological activity of isocitrate dehydrogenase 1 (IDH1) may be administered as a single compound or in combination with another compound inhibits the biological activity of IDH1 or that has a different pharmacological activity.

As indicated above, pharmaceutically acceptable salts of the compounds are contemplated and also may be utilized in the disclosed methods. The term “pharmaceutically acceptable salt” as used herein, refers to salts of the compounds, which are substantially non-toxic to living organisms. Typical pharmaceutically acceptable salts include those salts prepared by reaction of the compounds as disclosed herein with a pharmaceutically acceptable mineral or organic acid or an organic or inorganic base. Such salts are known as acid addition and base addition salts. It will be appreciated by the skilled reader that most or all of the compounds as disclosed herein are capable of forming salts and that the salt forms of pharmaceuticals are commonly used, often because they are more readily crystallized and purified than are the free acids or bases.

Acids commonly employed to form acid addition salts may include inorganic acids such as hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of suitable pharmaceutically acceptable salts may include the sulfate, pyrosulfate, bisulfate, sulfite, bisulfate, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleat-, butyne-.1,4-dioate, hexyne-1,6-dioate, benzoate, chlorobenzoate, methylbenzoate, hydroxybenzoate, methoxybenzoate, phthalate, xylenesulfonate, phenylacetate, phenylpropionate, phenylbutyrate, citrate, lactate, α-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Bases useful in preparing such salts include sodium hydroxide, potassium hydroxide, ammonium hydroxide, potassium carbonate, sodium carbonate, sodium bicarbonate, potassium bicarbonate, calcium hydroxide, calcium carbonate, and the like.

The particular counter-ion forming a part of any salt of a compound disclosed herein is may not be critical to the activity of the compound, so long as the salt as a whole is pharmacologically acceptable and as long as the counter-ion does not contribute undesired qualities to the salt as a whole. Undesired qualities may include undesirably solubility or toxicity.

Pharmaceutically acceptable esters and amides of the compounds can also be employed in the compositions and methods disclosed herein. Examples of suitable esters include alkyl, aryl, and aralkyl esters, such as methyl esters, ethyl esters, propyl esters, dodecyl esters, benzyl esters, and the like. Examples of suitable amides include unsubstituted amides, monosubstituted amides, and disubstituted amides, such as methyl amide, dimethyl amide, methyl ethyl amide, and the like.

In addition, the methods disclosed herein may be practiced using solvate forms of the compounds or salts, esters, and/or amides, thereof. Solvate forms may include ethanol solvates, hydrates, and the like.

The pharmaceutical compositions may be utilized in methods of treating a disease or disorder associated with the biological activity of isocitrate dehydrogenase 1 (IDH1). As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration.

As used herein the term “effective amount” refers to the amount or dose of the compound, upon single or multiple dose administration to the subject, which provides the desired effect in the subject under diagnosis or treatment. The disclosed methods may include administering an effective amount of the disclosed compounds (e.g., as present in a pharmaceutical composition) for treating a disease or disorder associated with biological activity of IDH1.

An effective amount can be readily determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

A typical daily dose may contain from about 0.01 mg/kg to about 100 mg/kg (such as from about 0.05 mg/kg to about 50 mg/kg and/or from about 0.1 mg/kg to about 25 mg/kg) of each compound used in the present method of treatment.

Compositions can be formulated in a unit dosage form, each dosage containing from about 1 to about 500 mg of each compound individually or in a single unit dosage form, such as from about 5 to about 300 mg, from about 10 to about 100 mg, and/or about 25 mg. The term “unit dosage form” refers to a physically discrete unit suitable as unitary dosages for a patient, each unit containing a predetermined quantity of active material calculated to produce the desired therapeutic effect, in association with a suitable pharmaceutical carrier, diluent, or excipient.

Oral administration is an illustrative route of administering the compounds employed in the compositions and methods disclosed herein. Other illustrative routes of administration include transdermal, percutaneous, intravenous, intramuscular, intranasal, buccal, intrathecal, intracerebral, or intrarectal routes. The route of administration may be varied in any way, limited by the physical properties of the compounds being employed and the convenience of the subject and the caregiver.

As one skilled in the art will appreciate, suitable formulations include those that are suitable for more than one route of administration. For example, the formulation can be one that is suitable for both intrathecal and intracerebral administration. Alternatively, suitable formulations include those that are suitable for only one route of administration as well as those that are suitable for one or more routes of administration, but not suitable for one or more other routes of administration. For example, the formulation can be one that is suitable for oral, transdermal, percutaneous, intravenous, intramuscular, intranasal, buccal, and/or intrathecal administration but not suitable for intracerebral administration.

The inert ingredients and manner of formulation of the pharmaceutical compositions are conventional. The usual methods of formulation used in pharmaceutical science may be used here. All of the usual types of compositions may be used, including tablets, chewable tablets, capsules, solutions, parenteral solutions, intranasal sprays or powders, troches, suppositories, transdermal patches, and suspensions. In general, compositions contain from about 0.5% to about 50% of the compound in total, depending on the desired doses and the type of composition to be used. The amount of the compound, however, is best defined as the “effective amount”, that is, the amount of the compound which provides the desired dose to the patient in need of such treatment. The activity of the compounds employed in the compositions and methods disclosed herein are not believed to depend greatly on the nature of the composition, and, therefore, the compositions can be chosen and formulated primarily or solely for convenience and economy.

Capsules are prepared by mixing the compound with a suitable diluent and filling the proper amount of the mixture in capsules. The usual diluents include inert powdered substances (such as starches), powdered cellulose (especially crystalline and microcrystalline cellulose), sugars (such as fructose, mannitol and sucrose), grain flours, and similar edible powders.

Tablets are prepared by direct compression, by wet granulation, or by dry granulation. Their formulations usually incorporate diluents, binders, lubricants, and disintegrators (in addition to the compounds). Typical diluents include, for example, various types of starch, lactose, mannitol, kaolin, calcium phosphate or sulfate, inorganic salts (such as sodium chloride), and powdered sugar. Powdered cellulose derivatives can also be used. Typical tablet binders include substances such as starch, gelatin, and sugars (e.g., lactose, fructose, glucose, and the like). Natural and synthetic gums can also be used, including acacia, alginates, methylcellulose, polyvinylpyrrolidine, and the like. Polyethylene glycol, ethylcellulose, and waxes can also serve as binders.

Tablets can be coated with sugar, e.g., as a flavor enhancer and sealant. The compounds also may be formulated as chewable tablets, by using large amounts of pleasant-tasting substances, such as mannitol, in the formulation. Instantly dissolving tablet-like formulations can also be employed, for example, to assure that the patient consumes the dosage form and to avoid the difficulty that some patients experience in swallowing solid objects.

A lubricant can be used in the tablet formulation to prevent the tablet and punches from sticking in the die. The lubricant can be chosen from such slippery solids as talc, magnesium and calcium stearate, stearic acid, and hydrogenated vegetable oils.

Tablets can also contain disintegrators. Disintegrators are substances that swell when wetted to break up the tablet and release the compound. They include starches, clays, celluloses, algins, and gums. As further illustration, corn and potato starches, methylcellulose, agar, bentonite, wood cellulose, powdered natural sponge, cation-exchange resins, alginic acid, guar gum, citrus pulp, sodium lauryl sulfate, and carboxymethylcellulose can be used.

Compositions can be formulated as enteric formulations, for example, to protect the active ingredient from the strongly acid contents of the stomach. Such formulations can be created by coating a solid dosage form with a film of a polymer which is insoluble in acid environments and soluble in basic environments. Illustrative films include cellulose acetate phthalate, polyvinyl acetate phthalate, hydroxypropyl methylcellulose phthalate, and hydroxypropyl methylcellulose acetate succinate.

Transdermal patches can also be used to deliver the compounds. Transdermal patches can include a resinous composition in which the compound will dissolve or partially dissolve; and a film which protects the composition, and which holds the resinous composition in contact with the skin. Other, more complicated patch compositions can also be used, such as those having a membrane pierced with a plurality of pores through which the drugs are pumped by osmotic action.

As one skilled in the art will also appreciate, the formulation can be prepared with materials (e.g., actives excipients, carriers (such as cyclodextrins), diluents, etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

### Inhibitors of Isocitrate Dehydrogenase 1 (IDH1) and Uses Thereof

Disclosed are compounds, pharmaceutical compositions comprising the compounds, and methods of using the compounds and pharmaceutical compositions for treating a subject having or at risk for developing a disease or disorder associated with IDH1 biological activity. The disclosed compounds may inhibit the biological activity of IDH1. As such, the disclosed compounds and pharmaceutical compositions may be utilized in methods for treating a subject having or at risk for developing a disease or disorder that is associated with IDH1 activity which may be cell proliferative diseases and disorders such as cancer.

In one aspect of the current disclosure methods for treating a cell proliferative disease in a subject in need thereof are provided. In some embodiments, the methods comprise administering to the subject a compound with the formula:

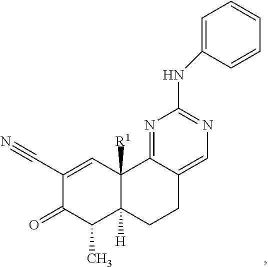

wherein R¹ is selected from methyl or phenyl.

In another aspect of the current disclosure, methods for treating a subject in need of treatment for a disease or disorder associated with expression of wild type isocitrate dehydrogenase (IDH1) are provided. In some embodiments, the methods comprise administering to the subject an effective amount of a therapeutic agent that inhibits the biological activity of IDH1.

As used herein, “expression of wild type isocitrate dehydrogenase 1 (IDH1)” refers to either the mRNA or protein expression of wild type IDH1 which can be measured using several methods known in the art. In some embodiments, “associated with expression of IDH1” refers to a disease or disorder that is characterized by the expression of IDH1 in affected cells. In some embodiments, IDH1 expression in affected cells is higher than similar, unaffected cells. In some embodiments, IDH1 expression in affected cells is about the same as IDH1 expression in similar, unaffected cells, but IDH1 may be critical to the survival or function of the affected cells. In some embodiments, IDH1 expression is lower than in similar unaffected cells, but IDH1 may be critical to the survival or function of the affected cells. Thus, in some embodiments, targeting IDH1 with the disclosed methods specifically impacts affected cells, while sparing unaffected cells. In some embodiments, “impacting” affected cells comprises one or more of the following: killing the affected cells, reducing the rate of growth of the affected cells, and potentiating the effects of another treatment modality, e.g., radiation therapy.

In some embodiments, the disclosed methods include treating a subject in need of treatment for a disease or disorder associated with IDH1 activity. In the disclosed methods, the subject may be administered an effective amount of a therapeutic agent that inhibits the biological activity of IDH1.

In the disclosed methods, a subject in need thereof typically is administered a therapeutic agent that inhibits the biological activity of IDH1. In some embodiments, the therapeutic agent inhibits the oxidative decarboxylation activity (E.C.: 1.1.1.42) of IDH1. In some embodiments, radiation therapy is administered in addition to the therapeutic agent. In some embodiments, radiation therapy is administered subsequently to the therapeutic agent. In some embodiments, the therapeutic agent is administered multiple times. In some embodiments, radiation therapy is administered in addition to the therapeutic agent multiple times. In some embodiments, several rounds of radiation therapy are administered to the subject, wherein each round is subsequent to administration of the therapeutic agent, or the therapeutic agent is administered continuously.

Suitable therapeutic agents for use in the disclosed methods may include, but are not limited to, a compound having a formula

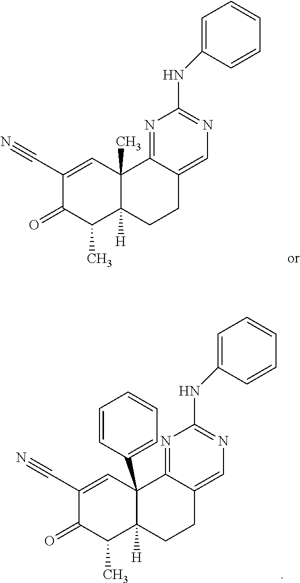 [SMILES: C[C@@H]1C(=O)C(C#N)=C[C@]2(C)c3nc(Nc4ccccc4)ncc3CC[C@@H]12 ; C[C@@H]1C(=O)C(C#N)=C[C@]2(c3ccccc3)c3nc(Nc4ccccc4)ncc3CC[C@@H]12]

In some embodiments of the disclosed methods, the therapeutic agent administered to the subject may be the compound having the formula:

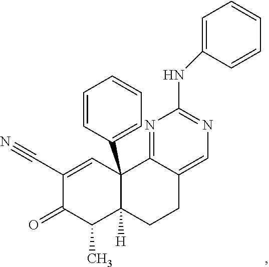 [SMILES: C[C@@H]1C(=O)C(C#N)=C[C@]2(c3ccccc3)c3nc(Nc4ccccc4)ncc3CC[C@@H]12]

otherwise referred to as (6aS,7S,10aR)-2-anilino-7-methyl-8-oxo-10a-phenyl-5,6,6a,7-tetrahydrobenzo[h] quinazoline-9-carbonitrile, or WT-IDHi-13, or 13i.

In some embodiments of the disclosed methods, the therapeutic agent administered to the subject may be the compound having the formula:

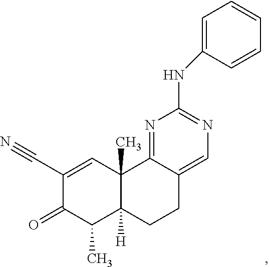 [SMILES: C[C@@H]1C(=O)C(C#N)=C[C@]2(C)c3nc(Nc4ccccc4)ncc3CC[C@@H]12]

otherwise referred to as (6aS,7S,10aR)-2-anilino-7,10a-dimethyl-8-oxo-5,6,6a,7-tetrahydrobenzo[h] quinazoline-9-carbonitrile.

The disclosed methods also may be performed in order to potentiate the effects of radiation therapy. As used herein, “potentiate” refers to the ability of one treatment to increase the power, effect, or likelihood of response to another therapy, e.g., radiation therapy (RT).

There are several advantages to targeting IDH1. 13i is the only wt-IDH1-specific small molecule inhibitor available.

Several companies developed inhibitors specific for the R132H mutant form of IDH1, including Agios (AG-120, AG-881; phase I), Bayer (BAY1436032; phase I initiated), Novartis (IDH305; phase I), GSK (GSK321, GSK864; discovery); and Eli Lilly (discovery).

For many of these compounds, the mechanism of inhibition has been elucidated at the molecular level by defining cocrystal structures of the inhibitor bound to enzymes by x-ray crystallography. Most inhibitors regulate enzyme activity allosterically, rather than competing for substrate binding to the active site. GSK321 and IDH305 inhibit mutant IDH1 by engaging an allosteric pocket formed when the enzyme adopts an open configuration. The inhibitor does not occupy the NADPH-binding site or engage the active center at Arg132, but instead one molecule of GSK321 binds to each monomer and induces structural changes in the tertiary structure of the enzyme, resulting in a catalytically inactive conformation. AG-881, the first dual-specific inhibitor targeted to both mutant IDH1 and IDH2, binds an allosteric pocket present in both enzymes and locks enzymes in an inactive conformation.

As these mt-IDH1-specific inhibitors recognize an allosteric site within the enzyme dimerization domain, many of these compounds show ‘off-target’ effect against wt-IDH1, as elucidated in cell-free enzyme repression assays. However, using GSK864 as an example, drug concentrations sufficient to impact growth and cell death in patient-derived HGG cells are in the high micromolar range, and in vivo effects are moderate. Similar effects with other IDH1 mutant compounds, in particular Agios' AG-881 (unpublished results), have been observed.

13i is a potent radiosensitizer and represents a novel therapeutic to improve HGG treatment outcomes.

RT has improved survival in multiple randomized trials for HGG, but around 80% of tumors recur within the high-dose RT field. The strategy of combining RT with wt-IDH1 inhibition builds upon the long and successful history of combining RT with anti-metabolites, e.g., inhibitors of the folate cycle or ribonucleotide reductase. Those approaches remain the standard-of-care treatment for many locally advanced malignancies, have recently shown clinical promise in the context of glioma, but can be associated with dose-limiting toxicity presumably because both cancers and normal tissues require these enzymes to mitigate RT-induced ROS and DNA double-strand breaks. This lack of selectivity is a major limitation of current anti-metabolite therapy. It is believed that this limitation is unlikely to apply to the inhibition of wt-IDH1, as wt-IDH1 has a higher maximal enzymatic activity than any other NADPH-producing enzymes in HGG, is the most differentially expressed NADPH producing enzyme in HGG compared to normal brain tissue, and the global ablation of wt-IDH1 does not impact normal organ physiology in mice.

13i target overexpressed wt-IDH1, a high-priority cancer target, in particular in HGG.

Using 13i, the Examples identify and credential wt-IDH1 as a druggable enzyme that contributes to RT treatment resistance in HGG.

Cancers up-regulate a variety of metabolic genes that conspire to reprogram tumor cell metabolism and support unabated growth and therapy resistance. Consequently, drug development efforts continue to focus on inhibiting a plethora of metabolic enzymes, many of which are being tested in combination with targeted and conventional chemo-and radiation therapy. These include compounds that target glucose transporters and enzymes implicated in glycolysis and fatty acid biosynthesis. Importantly, two aspects of the Examples set apart wt-IDH1 overexpression from induction of other metabolic enzymes: wt-IDH1 over-expression is highly prevalent in a variety of solid and systemic cancers, with IDH1 being the most differentially expressed NADPH producing enzyme in HGG tumors. This suggests that IDH1-generated NADPH is critical and likely rate limiting for maintaining lipid and ROS homeostasis to promote tumor cell growth and survival. In addition, lack of significant toxicity associated with wt-IDH1 inhibition is supported by phenotypical analysis of global wt-IDH1 K.O. mice. Associated results demonstrated that wt-IDH1 ablation does not impact normal cell development This is in agreement with our finding that pharmacological or genetic wt-IDH1 inactivation does not impact NADPH levels in normal cortical astrocytes1. These data suggest that genetic and pharmacological inhibition of cancer-associated wt-IDH1 has anti-proliferative and pro-apoptotic effects in tumor cells, due to elevated IDH1 expression and the development of an oncogenic dependency on IDH1 in cancer but not in normal cells. The therapeutic index is expected to be significant.

Harnessing the induction of ferroptosis via 13i treatment.

Ferroptosis is a recently discovered form of cell death that has not been associated with normal development, and consequently, has rapidly gained recognition as a paradigm shifting strategy to target cancer cells14. The Examples using 13i are first to credential the induction of ferroptosis as a tumor suppressive and therapeutically exploitable mechanism in wt-IDH1high HGG.

Targeting wt-IDH1 by 13i is expected to modulate the tumor-associated immune system.

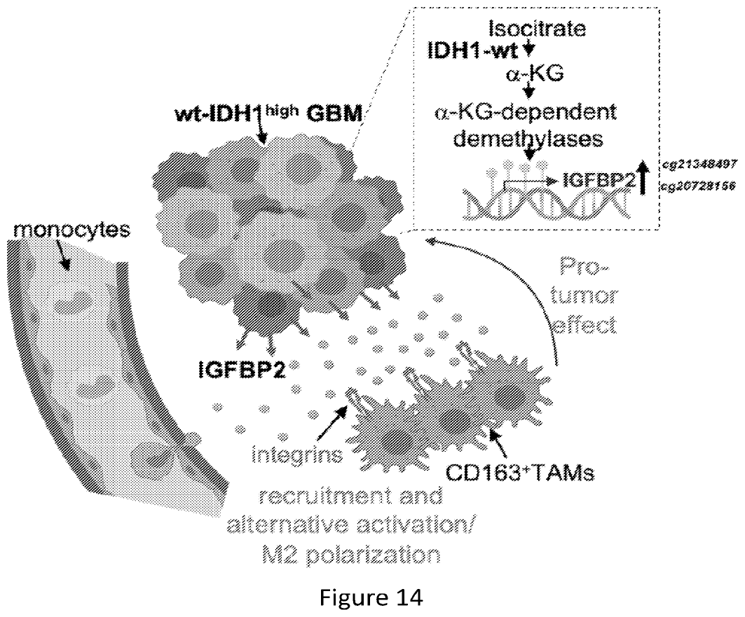

The Cancer Genome Atlas (TCGA) data was analyzed to identify wt-IDH1 gene expression relationships and IDH1-wt high expression was highly correlated with macrophage gene signatures. Subsequently, immune cell composition in syngeneic GBM engraftment models were examined and identified a substantial immunosuppressive (CD163+) tumor-associated macrophage (TAM) component, and a reduced CD8+/CD163+TAM ratio. Using array-based cytokine profiling of sera from mice bearing IDH1-wt high orthotopic GBM, Insulin Growth Factor Binding Protein 2 (IGFBP2), a potent oncogene and biomarker in a broad spectrum of cancers, including GBM, was identified as a secreted factor capable of promoting macrophage recruitment to tumor. IGFBP2 expression, in turn, was induced by alpha-KG-dependent demethylation of 2 CpG sites located in intron 1 of the IGFBP2 gene. These studies demonstrate that wt-IDH1 through alpha-KG production and the ensuing activation of demethylases regulates a key cytokine that is implicated in the recruitment of tumor-promoting macrophages (FIG. 14). It is believed that the anti-tumor effect of 13i is enhanced in immunocompetent models, due to its impact on wt-IDH1 paracrine signaling and its effect on tumor-associated macrophages.

### EXAMPLES

The following Examples are illustrative and should not be interpreted to limit the scope of the claimed subject matter.

### Example 1—Wild Type IDH1 Inhibition using a First-in-Class Covalent and Brain-Penetrant Small Molecule Inhibitor for Ferroptosis Induction in High-Grade Glioma

Elevated Expression of wt-IDH1 in HGG.

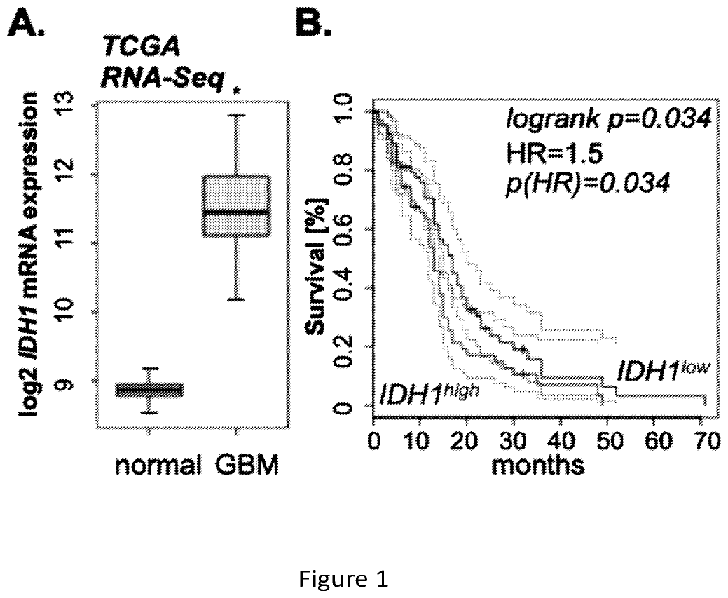

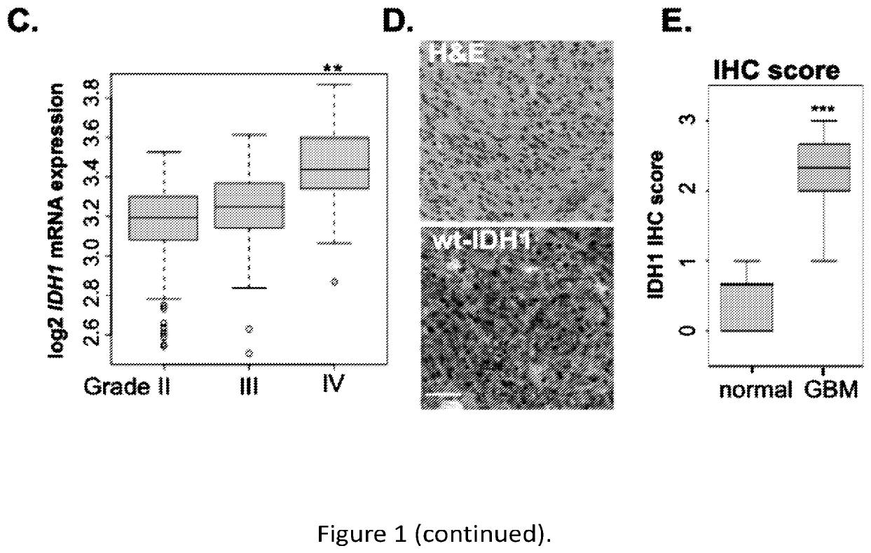

wt-IDH1 catalyzes the oxidative decarboxylation of isocitrate to alpha-ketoglutarate, NADPH and CO₂. Expanding the understanding of pathogenic IDH1 function beyond well-defined roles of neomorphic mutations, recent studies (see Stegh and colleagues, Cell Reports, 20171; Wahl and colleagues, Cancer Research, 20166) revealed that ⅔ of HGG overexpress wt-IDH1, as defined by having a fold change (FC)>1.5 in comparison to normal brain tissue (FIG. 1A). IDH1R132H point mutation and wt-IDH1 transcriptional up-regulation were mutually exclusive in this analysis¹. These findings point to distinct subtypes of HGG: low and high expressers of wild-type IDH1 (wt-IDH1low, wt-IDH1high, respectively), and mt-IDH1 grade IV astrocytoma¹. HGG patients with high wt-IDH1 mRNA expression showed significantly reduced survival in comparison to patients whose tumors have low wt-IDH1 mRNA (FIG. 1B). Levels of wt-IDH1 mRNA varied according to tumor cellular differentiation, transcriptome-defined subclassification¹, and tumor grade (FIG. 1C). Reflecting increases in wt-IDH1 transcript level, elevated wt-IDH1 protein expression was also evident through immunohistochemical analysis (FIG. 1D-E).

wt-IDH1 Expression is Important for Tumor Growth In Vivo.

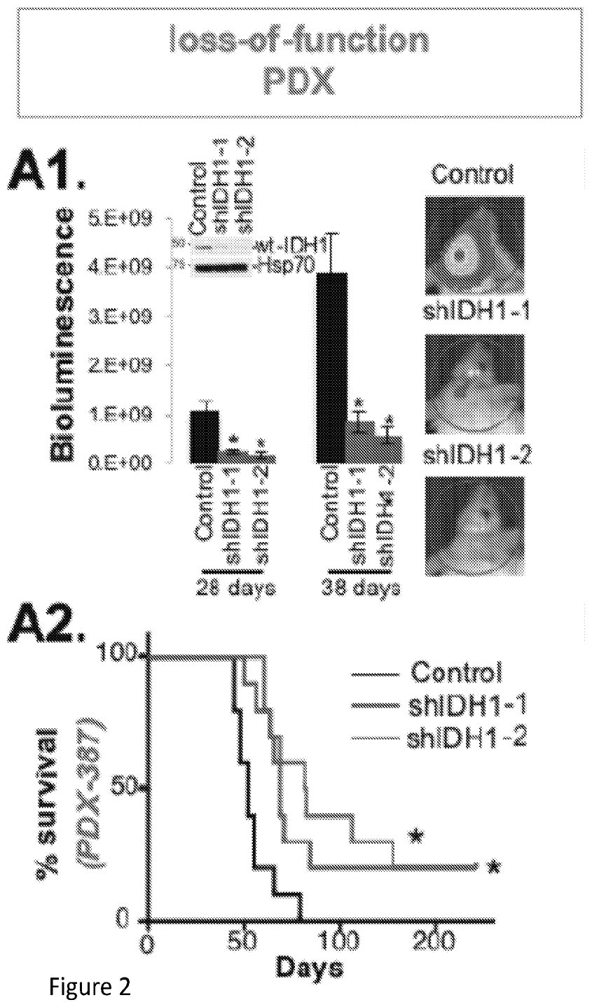

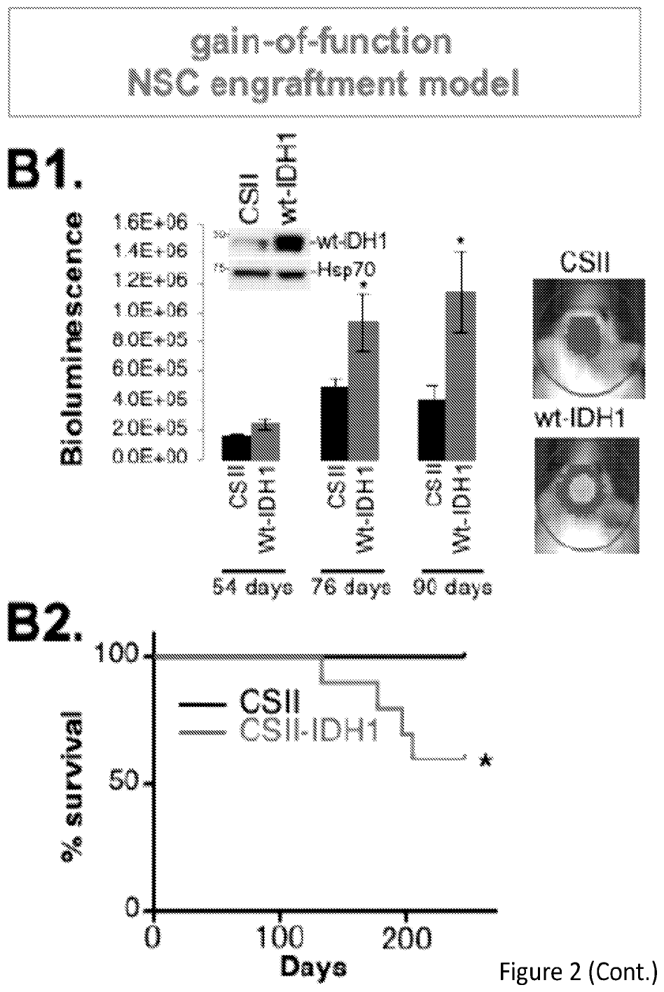

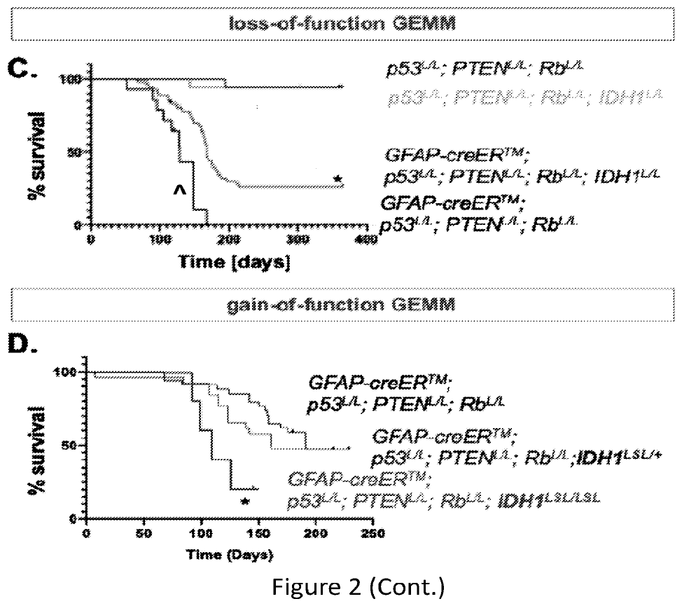

Knockdown of wt-IDH1 reduces the proliferation of HGG-derived glioma-initiating cells (GICs)¹, and knockdown cells grow more slowly as orthotopic xenografts, in comparison to cells without wt-IDH1 knockdown, following intracranial injection in NOD-SCID mice (FIG. 2A). Conversely, overexpression of wt-IDH1 in luciferase-expressing neural stem cells null for p53 and PTEN increases tumor formation and growth following intracranial injection into syngeneic mouse hosts and resulted in reduced animal survival (FIG. 2B)¹. To model somatic loss of wt-IDH1 in adult hosts, a conditional wt-IDH1 knockout allele was generated. Mice carrying the IDH1L allele were intercrossed with a triple tumor suppressor mouse, GFAP-cre-ERTT2;p53L/L;PTENL/L;RbL/L mice, developed by Baker and colleagues, which showed striking phenotypic similarity to human diffuse astrocytoma, and recapitulated the genetic diversity of the human disease². Kaplan-Meyer survival analysis indicated a significant increase in tumor-free survival in a genetically engineered mouse model (GEMM) that lacks wt-IDH1 expression (FIG. 2C, compare curve labelled with*and curve labelled with \hat{ }). To model CNS-specific increased wt-IDH1 expression, CRISPR/Cas9 genome editing technology was used for the knock-in of a murine wt-IDH1 transgene into the ROSA26 locus. The targeting vector contains a lox-STOP-lox (LSL)-controlled CAG promoter that drives the expression of a murine wt-IDH1-T2A-tdTomato-polyA fusion. IDH1LSL/LSL mice were subsequently crossed with GFAP-cre-ERTT2;p53L/L; PTENL/L;RbL/L mice, and wt-IDH1 induction upon oral gavage with tamoxifen was confirmed by western blotting of whole brain lysates. As shown in FIG. 2D, similar to NSC explant models, increase in wt-IDH1 expression reduces overall GEMM survival.

### Wt-IDH1 Promotes De Novo SFA and MUFA Biosynthesis.

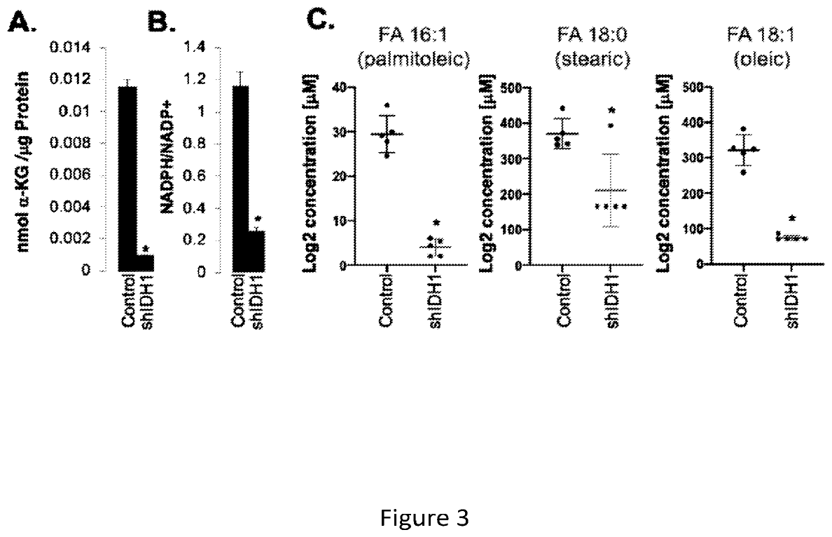

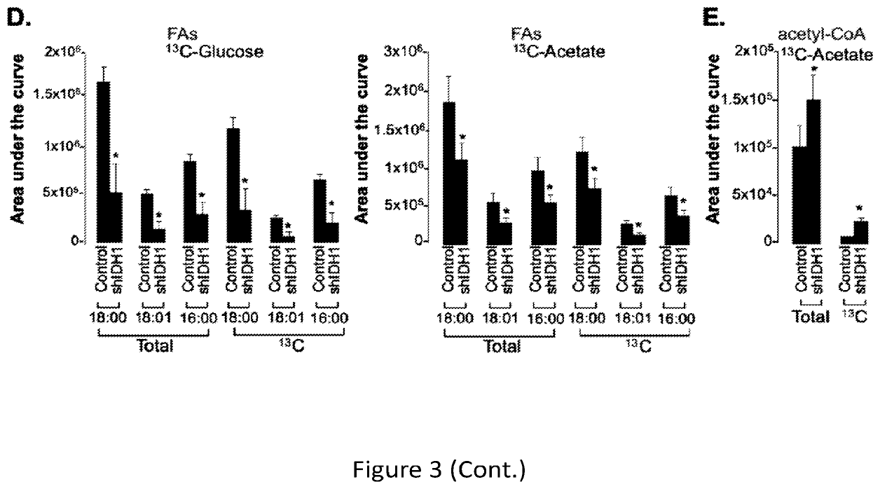

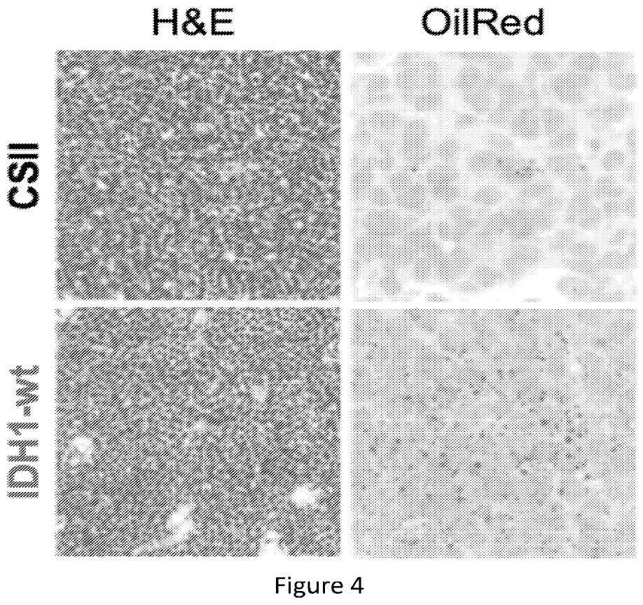

Studies in liver and adipose cells and tissue revealed that wt-IDH1 controls lipid metabolism due to its ability to produce cytosolic NADPH, the rate-limiting factor for lipogenesis^{3,4}. Thus, the effect of wt-IDH1 on anaplerotic flux was explored, in particular lipid biosynthesis, by performing targeted metabolomic studies. Patient-derived GICs modified for stable wt-IDH1 knockdown had significantly reduced alpha-ketoglutarate (FIG. 3A) and NADPH levels (FIG. 3B). Using LC-MS-based quantification of free fatty acid species and uniformly ¹³C6-labeled glucose and ¹³C2 acetate tracers, one finds the reduction in the NADPH/NADP+ ratio to be associated with diminished de novo saturated (C16:0; C18:0) and mono-unsaturated fatty acid biosynthesis (C16:1; C18:1) (FIG. 3C-D). Under conditions of hypoxia⁵, and anchorage-independent tumor spheroid growth⁶, wt-IDH1 can promote the reductive formation of citrate from glutamine by catalyzing the conversion of alpha-ketoglutarate to isocitrate (the “reverse” reaction). Citrate can subsequently be converted to acetyl-CoA (coenzyme A) and then malonyl-CoA, the carbon precursors for de novo lipogenesis. To determine whether wt-IDH1, under normoxic conditions examined here, can promote anaplerotic replacement of acetyl-CoA by stimulating alpha-ketoglutarate production (via “forward reaction”), ¹³C-label incorporation into acetyl-CoA was analyzed. It was found that GICs expressing wt-IDH1 targeting shRNA exhibited elevated levels of 13C2-labeled acetyl-CoA (FIG. 3E), suggesting that acetyl-CoA accumulates in wt-IDH1-compromised cells, as it cannot be used for de novo fatty acid synthesis due to limited cytosolic NADPH availability. Consistently, the decrease of fatty acid biosynthesis in GICs with stable knockdown of wt-IDH1 corresponded to increased numbers of lipid droplets in wt-IDH1 overexpressing PDX tumors in vivo, as indicated by Oil Red O staining (FIG. 4).

### Wt-IDH1-Derived NADPH Promotes Antioxidant-Mediated RT Resistance

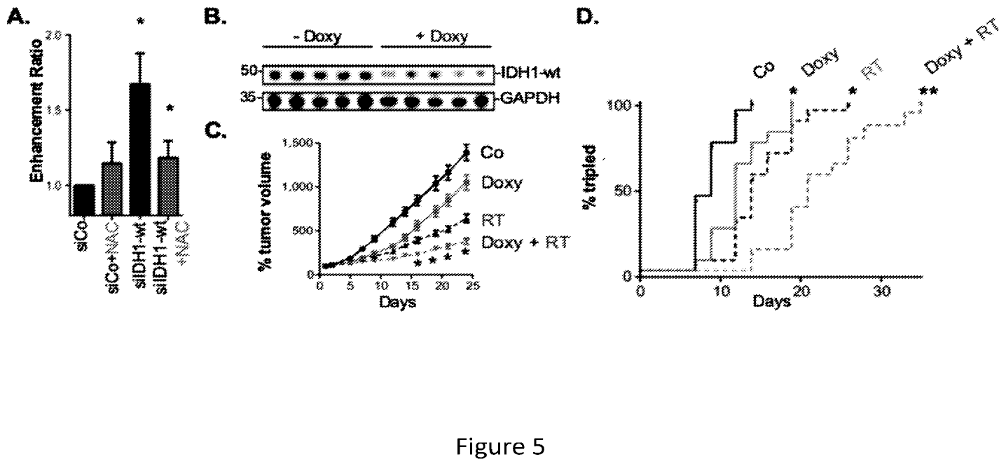

Knockdown of wt-IDH1 in 3 IDH1/2-wt, RT resistant cell lines with differing p53 mutational status (U87 p53wt, A172 p53mut, U138 p53mut) sensitized each of these cell lines to RT with enhancement ratios between 1.3 and 1.6, as determined in clonogenic assays (FIG. 5A, compare siDH1-wt vs. siCo), similar to the radiosensitization typically induced by temozolomide^{7,8}. Knockdown of wt-IDH1 decreased HGG cell line NADPH concentrations by 50-60%, and similarly depleted numerous NADPH-dependent metabolites, including the antioxidant glutathione (GSH). Consistent with these observations, the radiosensitization conferred by wt-IDH1 knockdown was mediated by increased ROS as it was nearly completely reversed by incubation with the antioxidant precursor N-acetyl cysteine (NAC) (FIG. 5A). Both control and wt-IDH1 knockdown cells had identical cell cycle profiles, suggesting that observed changes in metabolites are due to restricted NADPH production by wt-IDH1 rather than cell-cycle effects⁶. To determine whether combining RT and wt-IDH1 knockdown had beneficial anti-HGG effects in vivo, we established flank xenografts carrying a doxycycline (doxy)-inducible shRNA directed against wt-IDH1. A flank model was used due to the variable CNS penetrance of doxy. Immunoblotting confirmed that administration of doxy had the intended effects on wt-IDH1 expression in both inducible shIDH1 and shCo xenografts (FIG. 5B). Both radiation alone and doxycycline alone significantly slowed tumor growth compared with untreated tumors as analyzed using a linear mixed effects model (FIG. 5C). Combined doxy and RT significantly slowed tumor growth compared with either treatment alone (FIG. 5C), and supradditively increased the time to tumor tripling (median, 21 days) compared with RT alone (14 days), doxy alone (12 days), or no treatment (9 days, FIG. 5D)⁶.

IDH1i-13 is a First-in-Class Covalent Inhibitor of wt-IDH1.

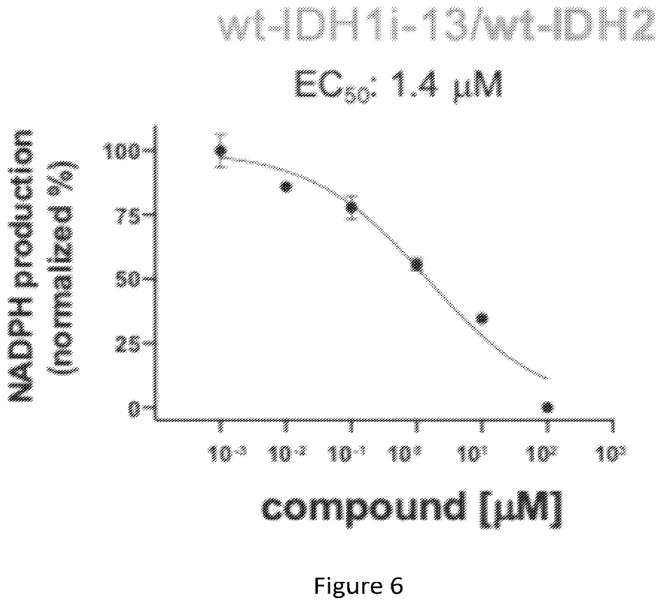

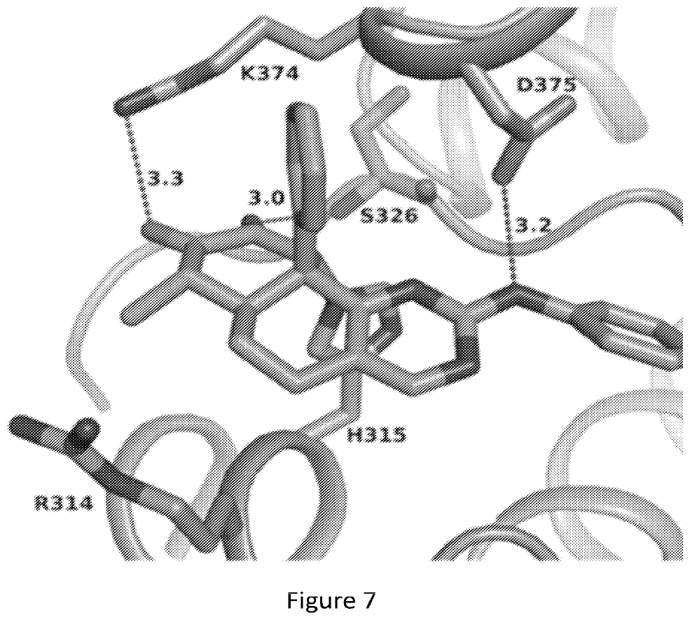

The compound 13i was developed using a high-throughput screen of a proprietary library, which encompasses 750,000 compounds. During the screen, the investigators identified a hit series, centered around compound wt-IDH1i-1, which contained a large cluster of α,β-unsaturated enones⁹. As indicated by the wt-IDH1: wt-IDH1i-1 crystal structure, wt-IDH1-1i bound to a fully closed wt-IDH1 enzyme and competed with NADPH for access to the wt-IDH1 active site via formation of a covalent adduct and the reversible trapping of H315 located within the NAPDH binding site⁹. Covalent engagement with the NADPH pocket resulted in robust enzyme inhibition, as indicated by a cell-free EC50 of 49 nM. Blastp searches identified wt-IDH2 as the only NADPH-dependent enzyme that contained an NADPH binding site with significant similarity to the NADPH binding pocket found in wt-IDH1. Despite sequence similarities, as shown in FIG. 6, 13i was less efficient in inhibiting wt-IDH2 activity and displayed a 100-fold higher EC50 against recombinant wt-IDH2 compared to wt-IDH1. Of note, while H315 and D375 are conserved between wt-IDH1 and wt-IDH2, other important amino acids, including those adjacent to D375 that form a lipophilic groove (K374/L383/A378) required for 13i binding are different, and are likely to mediate compound selectivity. These data demonstrate robust selectivity of 13i against wt-IDH1. Improved potency due to the replacement of a methyl with a phenyl group addition at R1 is consistent with the filling of a lipophilic groove between K374/L383/A378 observed in wt-IDH1: inhibitor co-crystal structures and suggested a clear trajectory to the K260 residue of the opposing monomer (FIG. 7).

### Wt-IDH1 Inhibits Ferroptosis.

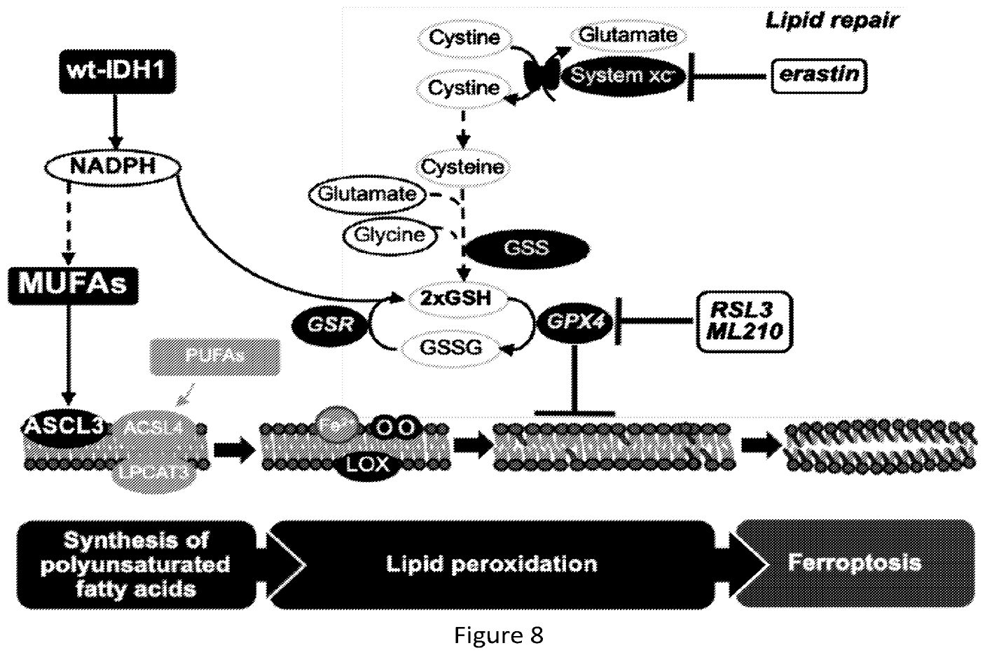

Increased lipid accumulation represents a unique metabolic state of cancer, including and especially in wt-IDH1high HGG^{11,12}. While required for growth, transformation and therapy resistance, increased lipid synthesis, in particular the synthesis of MUFAs, render cells less vulnerable to ferroptosis. On a molecular level, exogenous MUFAs, upon incorporation into cellular membranes, displace oxidizable PUFAs from the plasma membrane.¹³ PUFAs in turn, upon conversion into phospholipids by Acyl-CoA Synthetase Long Chain Family Member 4 (ACSL4) and Lysophosphatidylcholine Acyltransferase 3 (LPCAT3), are the substrates for lipid peroxidation by iron-dependent lipoxygenase (LOX) enzymes. Lipid peroxides decompose into reactive derivatives, including aldehydes and Michael acceptors, which can react with proteins and nucleic acids and trigger ferroptosis. The glutamate-cystine anti-porter xc-, glutathione synthetase (GSS), glutathione reductase (GSR) and the phospholipid peroxidase glutathione peroxidase 4 (GPX4) are critically important for lipid repair¹⁴. By exchange of glutamate at a 1:1 ratio, system xc-imports cystine, which upon conversion to cysteine is used for the synthesis of reduced glutathione (GSH) by GSS. GSH, which is replenished by GSR from oxidized glutathione (GSSG), is a co-factor for GPX4, which converts toxic lipid hydroperoxides to non-toxic lipid alcohols. Inactivation of GPX4 through depletion of GSH with erastin, a potent system xc—inhibitor, or with the direct GPX4 inhibitors (1S,3R)-RSL3 and ML210, results in lipid peroxidation that triggers ferroptosis (FIG. 8; references¹⁵⁻¹⁹). Ferroptosis is a form of cell death that has rapidly gained recognition as a paradigm shifting strategy to specifically target cancer cells and persister cells that are resistant to apoptosis induced by a broad spectrum of therapies, including RT and targeted therapies.^{14,20}

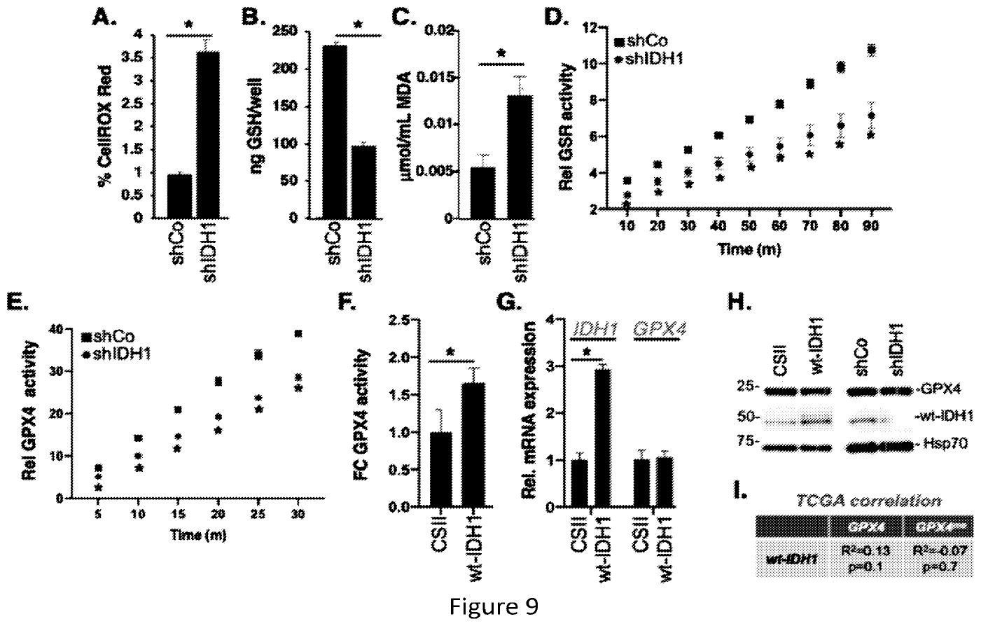

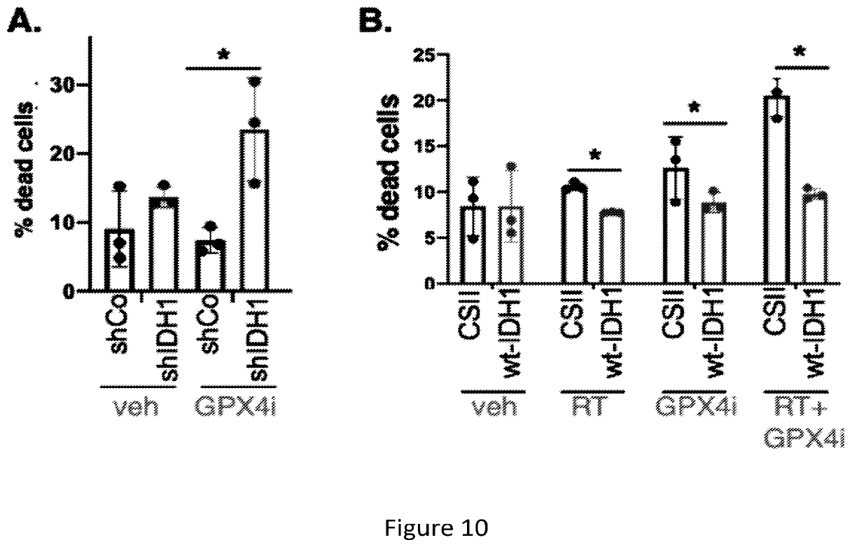

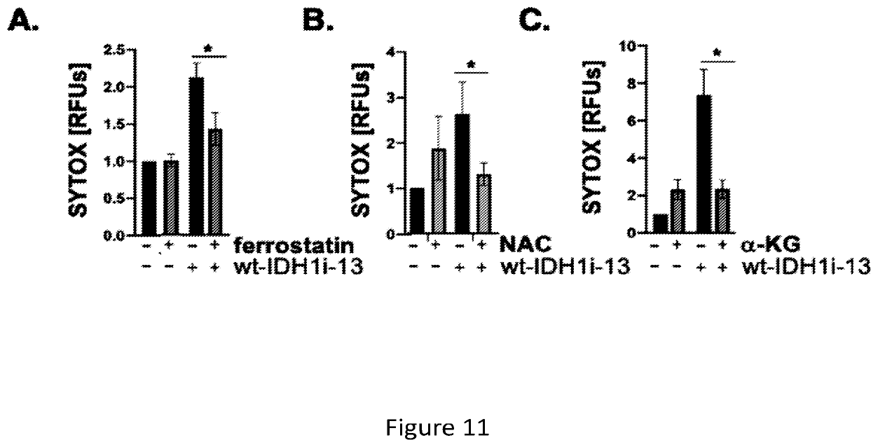

It was demonstrated that reduced NADPH production in patient-derived GICs with stable wt-IDH1 knockdown (see FIG. 3B) was associated with increased cellular ROS (FIG. 9A), diminished GSH level (FIG. 9B), and increased level of malondialdehyde, a marker for PUFA lipid peroxidation (MDA; FIG. 9C). The impact of wt-IDH1 on NADPH, ROS, GSH and lipid peroxide levels correlated with decreased GSR activity (FIG. 9D) in wt-IDH1 knockdown GICs, decreased GPX4 activity in shIDH1-expressing GICs (FIG. 9E) and increased GPX4 activity in wt-IDH1 overexpressing GICs (FIG. 9F). Of note, wt-IDH1 expression did not correlate with GPX4 mRNA or protein expression in patient-derived GICs (FIGS. 9G, H), or with GPX4 mRNA expression or GPX4 methylation in TCGA GBM (FIG. 9I). Furthermore, wt-IDH1 knockdown sensitized patient-derived GICs to cell death induced by the pro-ferroptosis GPX4 inhibitor RSL3 (FIG. 10A), while increased wt-IDH1 expression was associated with decreased plasma membrane rupture and cell death in response to RSL3 treatment alone, and in combination with, RT (FIG. 10B). Consistently, treatment of HGG glioma cells with 13i induced plasma membrane rupture, as indicated by increased SYTOX green positivity compared to vehicle control (FIG. 11). Co-treatment with ferrostatin (FIG. 11A) or NAC (FIG. 11B) rescued 13i treatment effect, similar to treatment with alpha-ketoglutarate (FIG. 11C), suggesting that pharmacologic wt-IDH1 inhibition induces ROS-mediated ferroptotic cell death.

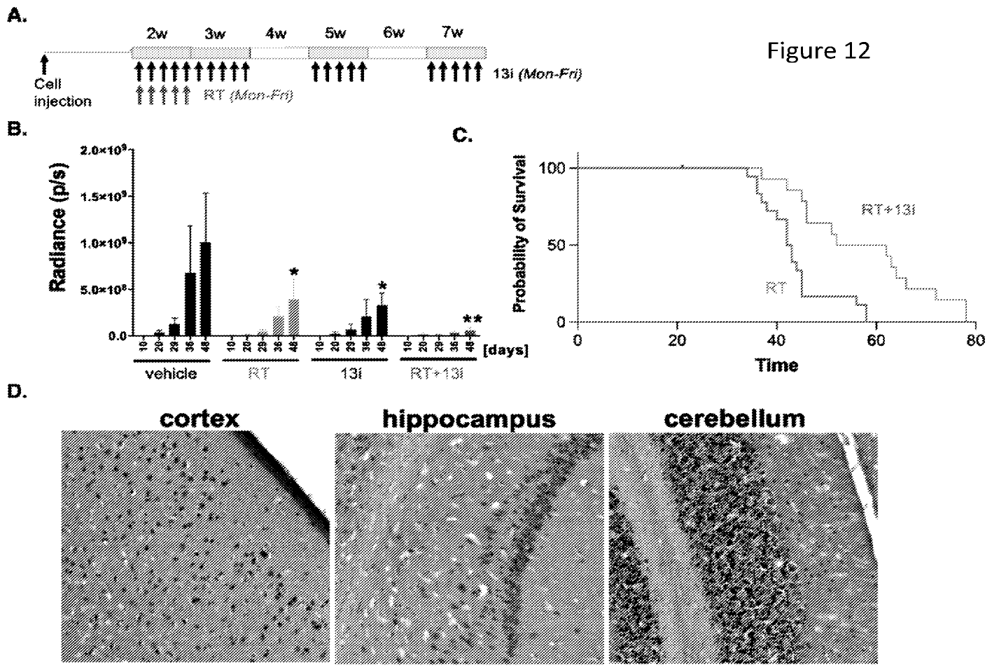

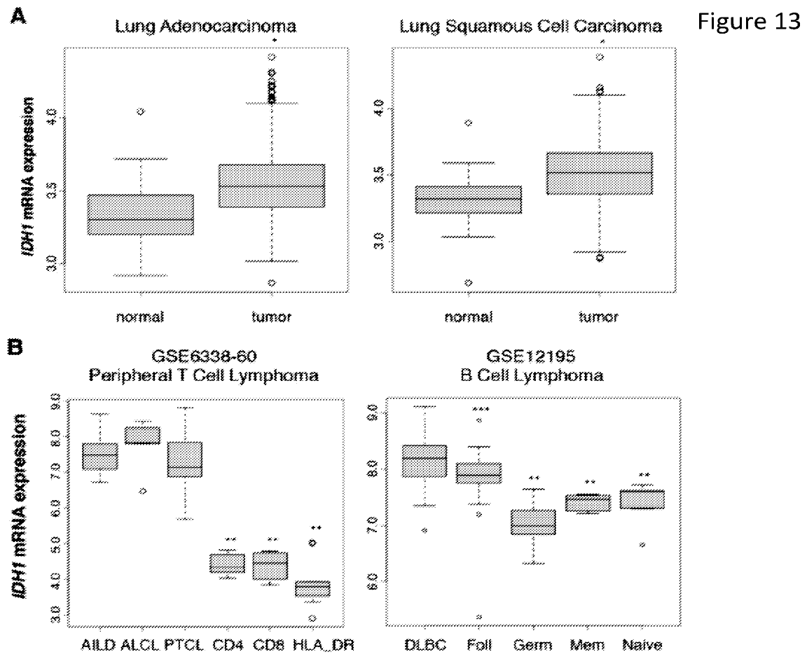

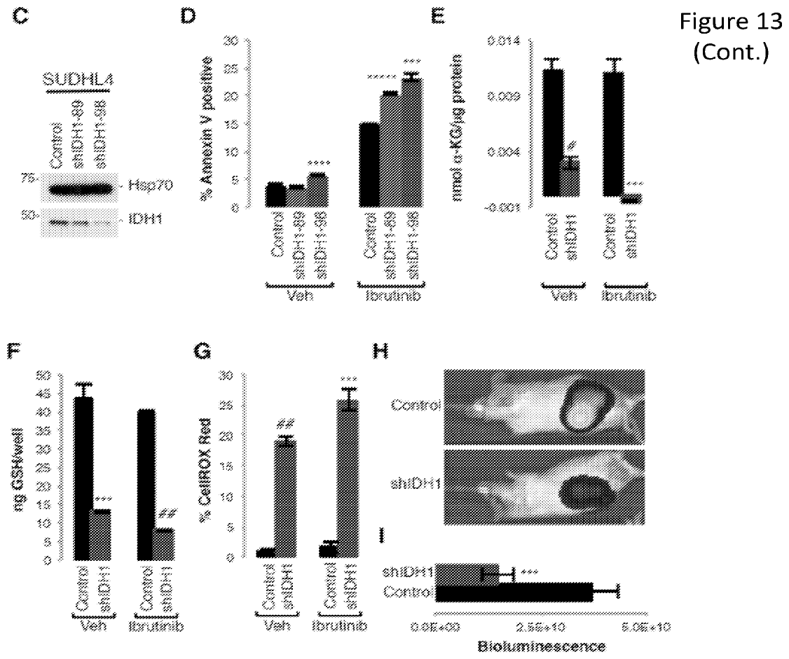

Importantly, the Examples reveal that the i.p. administration of 13i (FIG. 12A) resulted in enhancement of RT anti-tumor effect, as indicated by highly reduced tumor volume (FIG. 12B-C) and increased survival in 13i versus vehicle treated HGG PDX mice (FIG. 12D). Toxicity to normal brain structures was not observed, as indicated by H&E stainings of tumor-adjacent normal cortex, hippocampus and cerebellum (FIG. 12E).

These data demonstrate that wt-IDH1 enhances de novo MUFA biosynthesis and antagonizes ROS. Genetic and pharmacological inhibition of wt-IDH1 induces ferroptosis and promotes therapeutic effects of RT or GPX4 compromise. Further, intracranial pharmacological inhibition of wt-IDH1 is now possible using a brain-penetrant, first-in-class NADPH competitive inhibitor, which reduces HGG PDX progression, particularly when combined with RT.

### REFERENCES

1 Calvert, A. E. et al. Cancer-Associated IDH1 Promotes Growth and Resistance to Targeted Therapies in the Absence of Mutation. Cell Rep 19, 1858-1873, doi:10.1016/j.celrep.2017.05.014 (2017).PMC5564207
2 Chow, L. M. et al. Cooperativity within and among Pten, p53, and Rb pathways induces high-grade astrocytoma in adult brain. Cancer Cell 19, 305-316, doi:10.1016/j.ccr.2011.01.039 (2011).PMC3060664
3 Koh, H. J. et al. Cytosolic NADP+-dependent isocitrate dehydrogenase plays a key role in metabolism. The Journal of lipid biological chemistry 279, 39968-39974 , doi:10.1074/jbc.M402260200 (2004)
4 Shechter, I., Dai, P., Huo, L. & Guan, G. IDH1 gene transcription is sterol regulated and activated by SREBP-1a and SREBP-2 in human hepatoma HepG2 cells: evidence that IDH1 may regulate lipogenesis in hepatic cells. Journal of lipid research 44, 2169-2180, doi:10.1194/jlr.M300285-JLR200 (2003)
5 Metallo, C. M. et al. Reductive glutamine metabolism by IDH1 mediates lipogenesis under hypoxia. Nature 481, 380-384, doi: 10.1038/nature10602 (2012).3710581
6 Jiang, L. et al. Reductive carboxylation supports redox homeostasis during anchorage-independent growth. Nature 532, 255-258, doi:10.1038/nature17393 (2016).PMC4860952
7 Chakravarti, A. et al. Temozolomide-mediated radiation enhancement in glioblastoma: a report on underlying mechanisms. Clin Cancer Res 12, 4738-4746, doi:10.1158/1078-0432.CCR-06-0596 (2006)
8 Choi, E. J. et al. Enhanced cytotoxic effect of radiation and temozolomide in malignant glioma cells: targeting PI3K-AKT-mTOR signaling, HSP90 and histone deacetylases. BMC Cancer 14, 17, doi:10.1186/1471-2407-14-17 (2014).PMC3910677
9 Jakob, C. G. et al. Novel Modes of Inhibition of Wild-Type Isocitrate Dehydrogenase 1 (IDH1): Direct Covalent Modification of His315. J Med Chem 61, 6647-6657, doi:10.1021/acs.jmedchem.8b00305 (2018)
10 Chung, C. et al. Integrated Metabolic and Epigenomic Reprograming by H3K27M Mutations in Diffuse Intrinsic Pontine Gliomas. Cancer Cell 38, 334-349 e339, doi:10.1016/j.ccell.2020.07.008 (2020).PMC7494613
11 Geng, F. & Guo, D. Lipid droplets, potential biomarker and metabolic target in glioblastoma. Intern Med Rev (Wash D C) 3, doi:10.18103/imr.v3i5.443 (2017).PMC5639724
12 Guo, D., Bell, E. H. & Chakravarti, A. Lipid metabolism emerges as a promising target for malignant glioma therapy. CNS Oncol 2, 289-299, doi:10.2217/cns. 13.20 (2013).PMC3804348
13 Magtanong, L. et al. Exogenous Monounsaturated Fatty Acids Promote a Ferroptosis-Resistant Cell State. Cell Chem Biol 26, 420-432 e429, doi:10.1016/j.chembiol.2018.11.016 (2019).PMC6430697
14 Stockwell, B. R., Jiang, X. & Gu, W. Emerging Mechanisms and Disease Relevance of Ferroptosis. Trends Cell Biol 30, 478-490, doi:10.1016/j.tcb.2020.02.009 (2020).PMC7230071
15 Eaton, J. K. et al. Selective covalent targeting of GPX4 using masked nitrile-oxide electrophiles. Nat Chem Biol 16, 497-506, doi:10.1038/s41589-020-0501-5 (2020).PMC7251976
16 Eaton, J. K., Ruberto, R. A., Kramm, A., Viswanathan, V. S. & Schreiber, S. L. Diacylfuroxans Are Masked Nitrile Oxides That Inhibit GPX4 Covalently. J Am Chem Soc 141, 20407-20415, doi:10.1021/jacs.9b10769 (2019)
17 Hangauer, M. J. et al. Drug-tolerant persister cancer cells are vulnerable to GPX4 inhibition. Nature 551, 247-250, doi:10.1038/nature24297 (2017).PMC5933935
18 Liu, H., Schreiber, S. L. & Stockwell, B. R. Targeting Dependency on the GPX4 Lipid Peroxide Repair Pathway for Cancer Therapy. Biochemistry 57, 2059-2060, doi:10.1021/acs.biochem.8b00307 (2018).PMC5962875
19 Zou, Y. et al. A GPX4-dependent cancer cell state underlies the clear-cell morphology and confers sensitivity to ferroptosis. Nat Commun 10, 1617, doi:10.1038/s41467-019-09277-9 (2019).PMC6453886
20 Viswanathan, V. S. et al. Dependency of a therapy-resistant state of cancer cells on a lipid peroxidase pathway. Nature 547, 453-457, doi:10.1038/nature23007 (2017).PMC5667900
21 Wahl, D. R. et al. Glioblastoma therapy can be augmented by targeting IDH1-mediated NADPH biosynthesis. Cancer research, doi:10.1158/0008-5472.CAN-16-2008 (2016)

### Example 2—IDH1-Wt^{high} GBM Tumors Show Distinct Immune Cell Composition

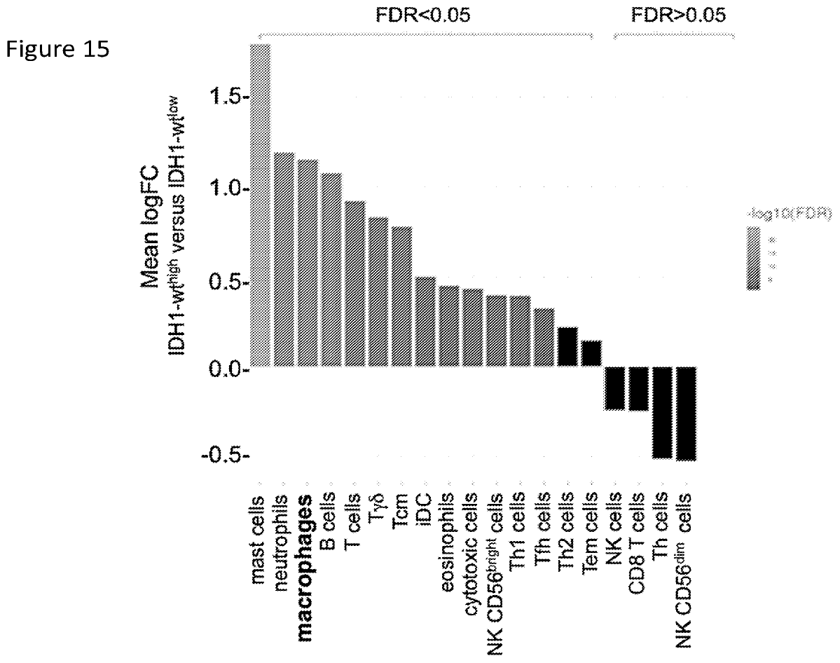

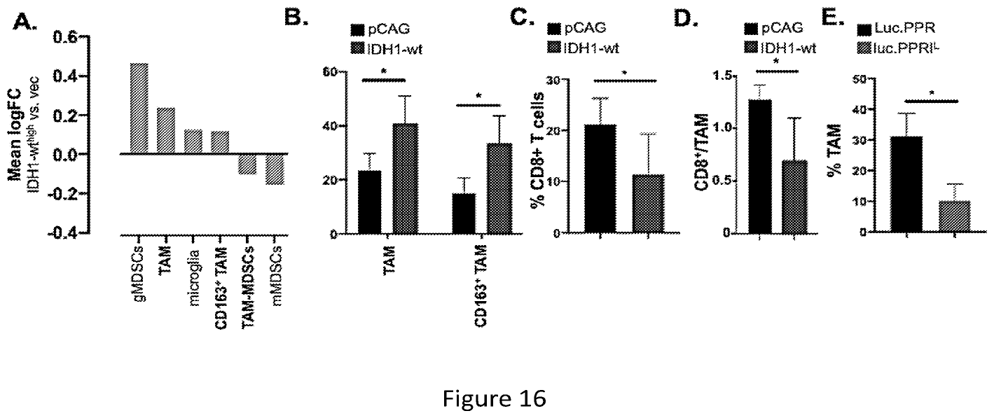

To identify immune cell populations in IDH1-wt^{high} GBM, TCGA GBM RNA-Seq data was analyzed for correlation between IDH1-wt transcript level and 19 types of immune cells using validated gene set signatures 1.2. GBM tumors were characterized as IDH1-wt^{low} (first quartile, n=38) and IDH1-wt^{high} (fourth quartile, n=38), and performed gene set enrichment analysis using GAGE 3. This analysis demonstrated that IDH1-wt level correlated with significant enrichment of innate immune cells, in particular macrophages, as well as with depletion of CD8⁺ T cells and cytotoxic natural killer (NK) cells (FIG. 15). Consistent with these human in silico data, IDH1-wt^{high} CT2A engraftment models showed higher TAM and CD163⁺TAM content, lower CD8+ T cell counts, and lower CD8⁺/TAM ratio in comparison to pCAG control CT2A.luc tumors (FIG. 16A-D). Conversely, evaluation of TAM content in 4-OHT-induced luc.PPR and luc.PPRI^{L} brains found decreased numbers of TAMs in mice with conditional loss of IDH1-wt (FIG. 16E). These data demonstrate that the utilized engraftment and transgenic models recapitulate IDH1-wt regulated TAM accumulation in GBM patients.

### Example 3—IDH1-Wt Induces IGFBP2 through α-KG-Dependent DNA Demethylation

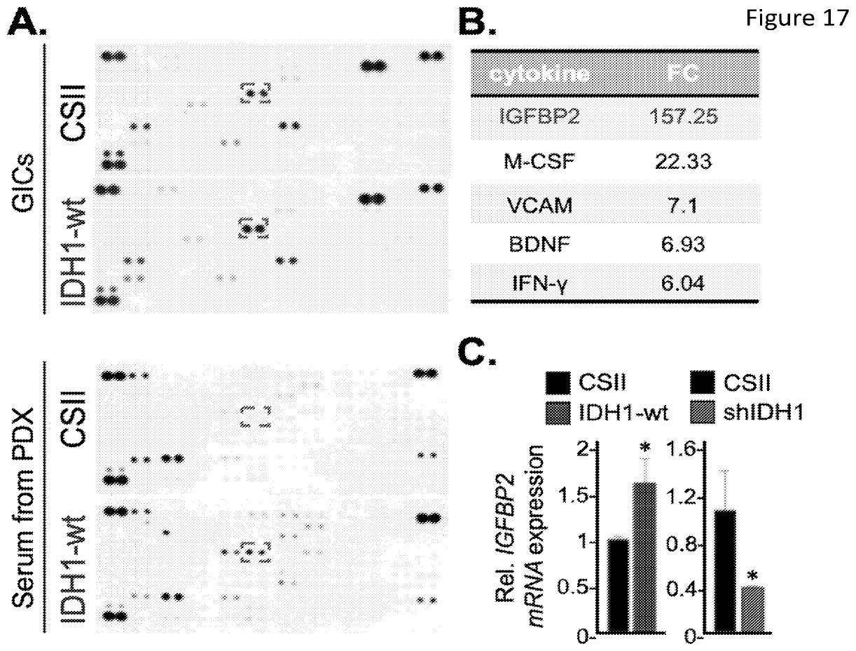

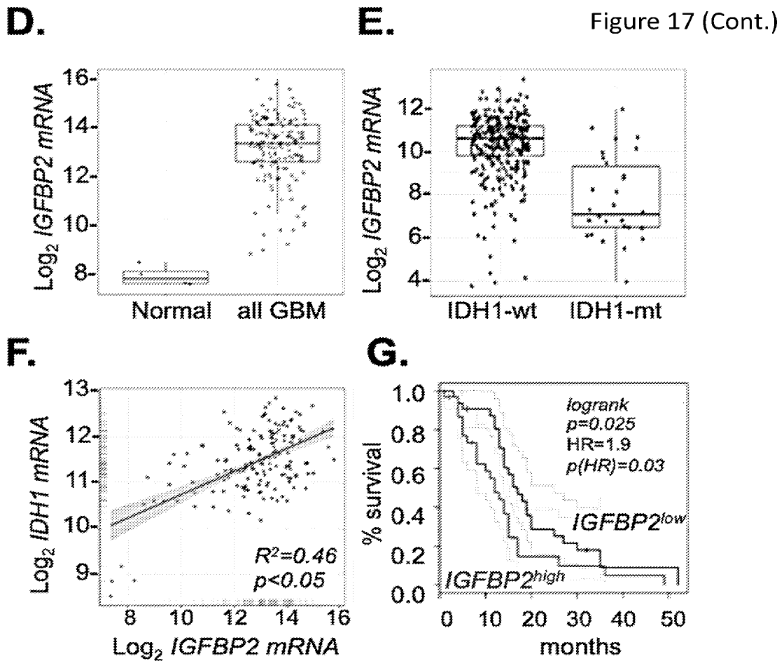

To gain insight regarding a potential molecular basis for IDH1-wt effect on immune cell recruitment, chemokine expression was analyzed in IDH1-wt^{high} patient-derived GICs in vitro and in serum of derivative PDX using cytokine arrays and RT-qPCR. When comparing cytokine abundance in cell culture supernatants of vector control versus GICs modified for expression of exogenous IDH1-wt, and in sera of mice bearing PDX established from the same cells, IGFBP2 was the chemokine showing the largest fold change in the supernatants and sera associated with IDH1-wt cells and xenografts, respectively (FIG. 17A, B). RT-qPCR of mRNA from GICs with stable knockdown or overexpression of IDH1-wt confirmed enhanced IGFBP2 transcript level in IDH1-wt overexpressing, and reduced IGFBP2 mRNA in IDH1-wt KD GICs relative to corresponding controls (FIG. 17C). Subsequently, IGFBP2 mRNA level was determined as being significantly upregulated in TCGA IDH1-wt GBM in comparison to IDH1-mt grade IV tumors and normal brain tissue (FIG. 17D, E), with IGFBP2 expression directly correlating with IDH1-wt mRNA level (FIG. 17F), and negatively impacting GBM patient survival (FIG. 17G).

IGFBP2 is a multifunctional protein that integrates oncogenic processes through Insulin growth factor (IGF)-dependent and-independent mechanisms ⁴. In gliomas, IGFBP2 expression has been used as a part of a 9-gene signature for predicting outcome ⁵⁻⁷. IGFBP2 promotes proliferation, migration and invasion, and is capable of initiating high-grade glioma formation, as determined in compound genetically engineered mouse models ^{8,9}. Proteomic analysis of GBM tumors identified IGFBP2 as a high-priority pro-inflammatory protein that is highly expressed in IDH1-wt GBM tumor cells ¹⁰. IGFBP2 binds to integrins ¹¹, e.g., integrin αᵥβ₅, which is the most highly expressed integrin on GBM tumor-infiltrating M0/M2 macrophages ¹². Integrin activation in macrophages results in downstream Src kinase activation, which triggers the phosphorylation and activation of FcγRIIB, an immune inhibitory receptor, and M2 alternative macrophage activation through engagement of yet to be defined signaling pathways ¹³

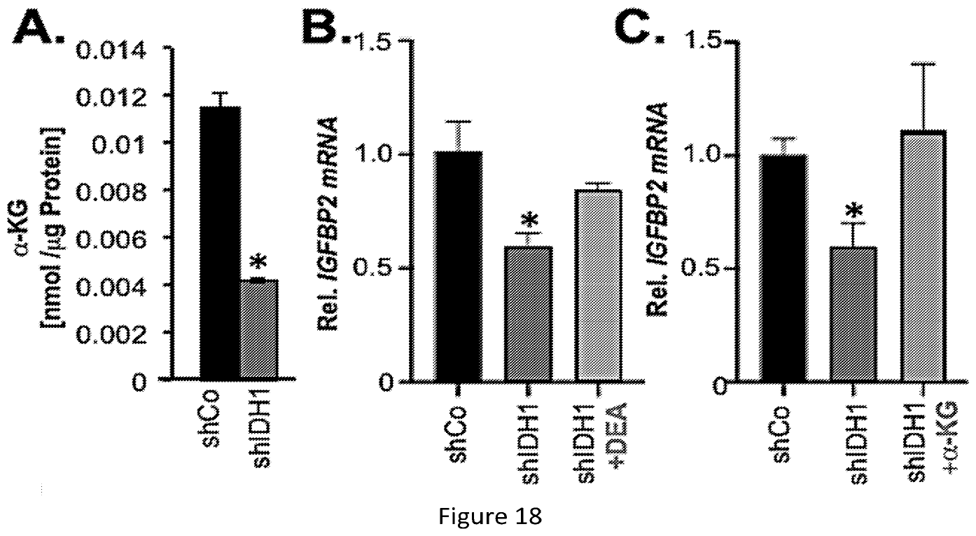

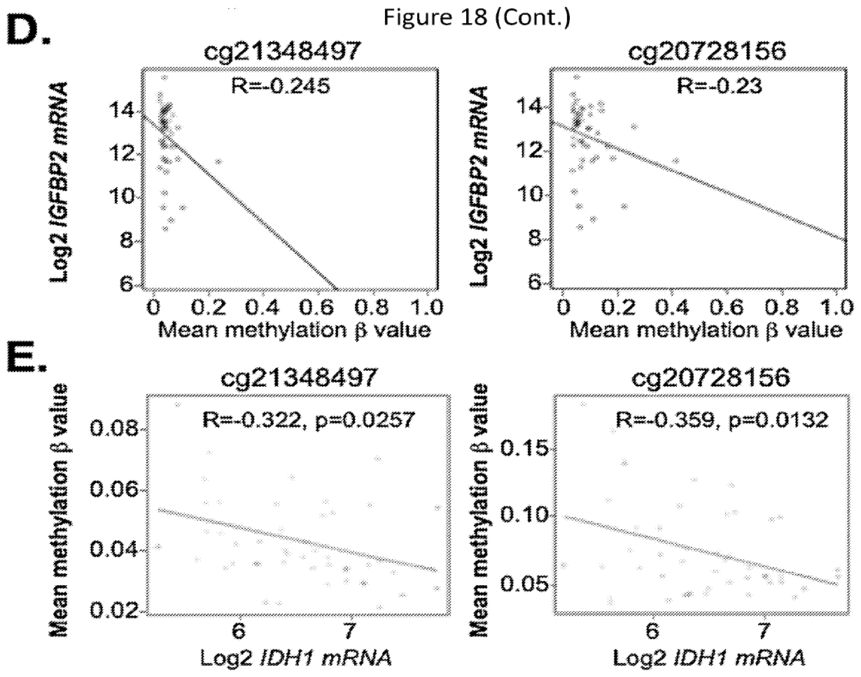

Next, whether IDH1-wt, through α-KG-dependent DNA demethylation, induces IGFBP2 transcription was tested. IDH1-wt produces α-KG (FIG. 18A, demonstrating that IDH1-wt KD is associated with reduced α-KG), which functions as co-factor for α-KG-dependent DNA dioxygenases that demethylate DNA ^{14,15}. GICs stably expressing IDH1-wt-specific shRNA were treated with the DNA demethylating agent decitabine or cell-permeable di-methyl-α-KG, and measured IGFBP2 mRNA level by RT-qPCR. Both treatments increased IGFBP2 mRNA level in IDH1-wt KD GICs to the level of shCo-infected cells (FIG. 18B, C). TCGA GBM RNA-Seq methylation array 450k datasets were analyzed and 2 methylation probes located within intron 1 of the IGFBP2 gene (cg21348497 and cg20728156) were identifiedd, whose methylation level inversely correlated with both IGFBP2 and IDH1-wt mRNA. (FIG. 18D, E). These data suggest that IDH1-wt through effect on α-KG-dependent dioxygenase activity reduces IGFPB2 methylation of intronic CpG sites and promotes IGFBP2 transcription.

### REFERENCES

1 Bindea, G. et al. Spatiotemporal dynamics of intratumoral immune cells reveal the immune landscape in human cancer. Immunity 39, 782-795, doi:10.1016/j.immuni.2013.10.003 (2013)
2 Engler, J. R. et al. Increased microglia/macrophage gene expression in a subset of adult and pediatric astrocytomas. PloS one 7, e43339, doi:10.1371/journal.pone.0043339 (2012).PMC3425586
3 Luo, W., Friedman, M. S., Shedden, K., Hankenson, K. D. & Woolf, P. J. GAGE: generally applicable gene set enrichment for pathway analysis. BMC bioinformatics 10, 161, doi:10.1186/1471-2105-10-161 (2009).PMC2696452
4 Li, T. et al. IGFBP2: integrative hub of developmental and oncogenic signaling network. Oncogene, doi:10.1038/s41388-020-1154-2 (2020)
5 Colman, H. et al. A multigene predictor of outcome in glioblastoma. Neuro-oncology 12, 49-57, doi:10.1093/neuonc/nop007 (2010).PMC2940562
6 Fuller, G. N. et al. Reactivation of insulin-like growth factor binding protein 2 expression in glioblastoma multiforme: a revelation by parallel gene expression profiling. Cancer research 59, 4228-4232 (1999)
Wang, H., Zhang, W. & Fuller, G. N. Tissue microarrays: applications in neuropathology research, diagnosis, and education. Brain pathology 12, 95-107, doi:10.1111/j.1750-3639.2002.tb00426.x (2002)
8 Dai, C. et al. PDGF autocrine stimulation dedifferentiates cultured astrocytes and induces oligodendrogliomas and oligoastrocytomas from neural progenitors and astrocytes in vivo. Genes & development 15, 1913-1925, doi:10.1101/gad.903001 (2001).PMC312748
9 Dunlap, S. M. et al. Insulin-like growth factor binding protein 2 promotes glioma development and progression. Proceedings of the National Academy of Sciences of the United States of America 104, 11736-11741, doi:10.1073/pnas.0703145104 (2007).PMC1913900
10 Klemm, F. et al. Interrogation of the Microenvironmental Landscape in Brain Tumors Reveals Disease-Specific Alterations of Immune Cells. Cell 181, 1643-1660 e1617, doi:10.1016/j.cell.2020.05.007 (2020)
11 Liu, Y., Song, C., Shen, F., Zhang, J. & Song, S. W. IGFBP2 promotes immunosuppression associated with its mesenchymal induction and FcgammaRIIB phosphorylation in glioblastoma. PloS one 14, e0222999, doi:10.1371/journal.pone.0222999 (2019). PMC6764691
12 Wei, J. et al. Osteopontin mediates glioblastoma-associated macrophage infiltration and is a potential therapeutic target. The Journal of clinical investigation 129, 137-149, doi:10.1172/JCI121266 (2019).PMC6307970
13 Espeli, M., Smith, K. G. & Clatworthy, M. R. FcgammaRIIB and autoimmunity. Immunol Rev 269, 194-211, doi:10.1111/imr. 12368 (2016)
14 Losman, J. A. & Kaelin, W. G., Jr. What a difference a hydroxyl makes: mutant IDH, (R)-2-hydroxyglutarate, and cancer.Genes & development 27, 836-852, doi: 10.1101/gad.217406.113 (2013).3650222
15 Tommasini-Ghelfi, S. et al. Cancer-associated mutation and beyond: The emerging biology of isocitrate dehydrogenases in human disease. Sci Adv 5, eaaw4543, doi:10.1126/sciadv.aaw4543 (2019).PMC6530995

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

1. A method for treating a cell proliferative disease in a subject in need thereof, the method comprising administering to the subject a compound with the formula: 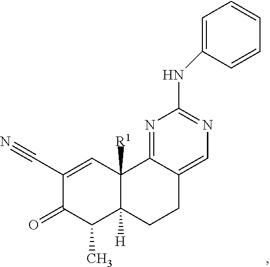
wherein R¹ is selected from methyl or phenyl.

2. The method of claim 1, wherein R¹ is phenyl.

3. The method of claim 1, wherein R¹ is methyl.

4. The method of claim 1, wherein the cell proliferative disease is cancer.

5. The method of claim 1, wherein the cell proliferative disease is brain cancer.

6. The method of claim 1, wherein the cell proliferative disease is selected from the group comprising: astrocytoma, oligodendroglioma, mixed glioma, and ependymoma.

7. The method of claim 1, wherein the cell proliferative disease is high grade glioma (HGG).

8. The method of claim 1, wherein the cell proliferative disease is characterized by a greater expression of wild type isocitrate dehydrogenase 1 (IDH1) than a control tissue.

9. The method of claim 1, wherein the method further comprises administering radiation therapy to the subject.

10. The method of claim 9, wherein the radiation therapy is administered subsequently to the compound.

11. A method for treating a subject in need of treatment for a disease or disorder associated with expression of wild type isocitrate dehydrogenase 1 (IDH1), the method comprising administering to the subject an effective amount of a therapeutic agent that inhibits the biological activity of IDH1.

12. The method of claim 11, wherein the therapeutic agent comprises the compound with the formula: 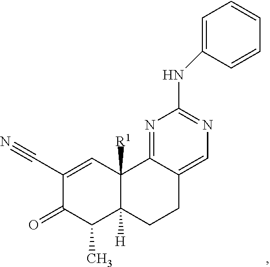
wherein R¹ is selected from methyl or phenyl.

13. The method of claim 12, wherein R¹ is methyl.

14. The method of claim 12, wherein R¹ is phenyl.

15. The method of claim 11, wherein the disease or disorder is a cell proliferative disease or disorder.

16. The method of claim 15, wherein the cell proliferative disease is cancer.

17. The method of claim 15, wherein the cell proliferative disease is brain cancer.

18. The method of claim 15, wherein the cell proliferative disease is selected from the group comprising astrocytoma, oligodendroglioma, mixed glioma, and ependymoma.

19. The method of claim 15, wherein the cell proliferative disease is high grade glioma (HGG).

20. The method of claim 11, wherein the method further comprises administering radiation therapy to the subject.

21. The method of claim 20, wherein the radiation therapy is administered subsequently to the compound.


SEQUENCE LISTING (2 sequences)

SEQ ID NO: 1 - protein, 414 aa, Homo sapiens
MSKKISGGSVVEMQGDEMTRIIWELIKEKLIFPYVELDLHSYDLGIENRDATNDQVTKDAAEAIKKHNVGVKCATITPDEKRVEEFKLKQMWKSPNGTIRNILGGTVFREAIICKNIPRLVSGWVKPIIIGRHAYGDQYRATDFVVPGPGKVEITYTPSDGTQKVTYLVHNFEEGGGVAMGMYNQDKSIEDFAHSSFQMALSKGWPLYLSTKNTILKKYDGRFKDIFQEIYDKQYKSQFEAQKIWYEHRLIDDMVAQAMKSEGGFIWACKNYDGDVQSDSVAQGYGSLGMMTSVLVCPDGKTVEAEAAHGTVTRHYRMYQKGQETSTNPIASIFAWTRGLAHRAKLDNNKELAFFANALEEVSIETIEAGFMTKDLAACIKGLPNVQRSDYLNTFEFMDKLGENLKIKLAQAKL

SEQ ID NO: 2 - DNA, 1113 bp, Homo sapiens
  source 1..1113: mol_type = other DNA
ctcatttttccctacgtggaattggatctacatagctatgatttaggcatagagaatcgtgatgccaccaacgaccaagtcaccaaggatgctgcagaagctataaagaagcataatgttggcgtcaaatgtgccactatcactcctgatgagaagagggttgaggagttcaagttgaaacaaatgtggaaatcaccaaatggcaccatacgaaatattctgggtggcacggtcttcagagaagccattatctgcaaaaatatcccccggcttgtgagtggatgggtaaaacctatcatcataggtcgtcatgcttatggggatcaatacagagcaactgattttgttgttcctgggcctggaaaagtagagataacctacacaccaagtgacggaacccaaaaggtgacatacctggtacataactttgaagaaggtggtggtgttgccatggggatgtataatcaagataagtcaattgaagattttgcacacagttccttccaaatggctctgtctaagggttggcctttgtatctgagcaccaaaaacactattctgaagaaatatgatgggcgttttaaagacatctttcaggagatatatgacaagcagtacaagtcccagtttgaagctcaaaagatctggtatgagcataggctcatcgacgacatggtggcccaagctatgaaatcagagggaggcttcatctgggcctgtaaaaactatgatggtgacgtgcagtcggactctgtggcccaagggtatggctctctcggcatgatgaccagcgtgctggtttgtccagatggcaagacagtagaagcagaggctgcccacgggactgtaacccgtcactaccgcatgtaccagaaaggacaggagacgtccaccaatcccattgcttccatttttgcctggaccagagggttagcccacagagcaaagcttgataacaataaagagcttgccttctttgcaaatgctttggaagaagtctctattgagacaattgaggctggcttcatgaccaaggacttggctgcttgcattaaaggtttacccaatgtgcaacgttctgactacttgaatacatttgagttcatggataaa